(19) United States
(12) Patent Application Publication
(10) Pub. No.: US 2010/0136546 A1
(43) Pub. Date: Jun. 3, 2010

(54) GENETIC MARKER FOR ADVERSE BEHAVIORAL CONDITIONS

(76) Inventors: Arthur Beaudet, Houston, TX (US); Shay Ben-Shachar, (US); Trilochan Sahoo, (US)

Correspondence Address: FULBRIGHT & JAWORSKI, LLP, 1301 MCKINNEY, SUITE 5100, HOUSTON, TX, 77010-3095, US

(21) Appl. No.: 12/551,313

(22) Filed: Aug. 31, 2009

Related U.S. Application Data
(60) Provisional application No. 61/093,319, filed on Aug. 30, 2008.

(51) Int. Cl. C12Q 1/68

(52) U.S. Cl. 435/6

(57) ABSTRACT

The present invention concerns genetic marker for adverse behavioral conditions. In particular aspects, the marker is present at chromosome 15q13 and comprises CHRNA7. In certain cases, the marker is a microdeletion or point mutation in CHRNA7.

This application claims priority to U.S. Provisional Patent Application Ser. No. 61/093,319, filed Aug. 30, 2008, which application is incorporated by reference herein in its entirety.

### STATEMENT REGARDING FEDERALLY SPONSORED RESEARCH OR DEVELOPMENT

This invention was made with government support under grant HD-37283 awarded by NIH. The United States Government has certain rights in the invention.

### TECHNICAL FIELD

The present invention generally relates to genetic diagnosis of particular behavior types, including adverse behavioral conditions. In particular, it relates to a genetic marker associated with individuals predisposed to being sex offenders, exhibit violent or aggressive criminal behavior, and/or exhibit psychopathic behavior, in certain cases.

### BACKGROUND OF THE INVENTION

The availability of the human genome sequence and the use of array-based hybridization to identify genomic copy number variations (CNVs) are catalyzing the rapid discovery of novel microdeletion and microduplication syndromes (Ballif et al., 2007; Ben-Shachar et al., 2008; Weiss et al., 2008). Presently, there is routine clinical utilization of genome-wide analysis of copy number generically termed chromosomal microarray analysis (CMA) or array genomic hybridization (AGH) to include both single nucleotide polymorphism (SNP)-based arrays and array comparative genomic hybridization (aCGH) without SNP analysis. The extensive distribution, high frequency, and gene content of benign and pathogenic CNVs across the genome have provided a new perspective regarding genomic polymorphism and “genomic disorders” (Sebat et al., 2004; Iafrate et al., 2004; Lupski, 1998). These CNVs often result in rearrangements that are mediated by nonallelic homologous recombination (NAHR) between large, highly homologous segmental duplications (low copy repeats). (Stankiewicz and Lupski, 2002). Many of the genomic regions that are flanked by segmental duplications have a high mutability for gain and loss of copy number and the frequency of de novo CNVs is much higher than that for de novo single base mutations (Lupski, 2007). Genomic disorders include well characterized microdeletion/duplication syndromes including DiGeorge/velocardiofacial syndrome (DGS/VCFS [MIM 188400/MIM 192430]), Williams-Beuren syndrome (WBS [MIM 194050]), and Smith-Magenis syndrome (SMS [MIM 182290]), and the reciprocal duplication syndromes for these three entities.

It has long been known that microscopically detectable chromosomal abnormalities cause mental retardation and less commonly autism (Vorstman et al., 2006) with virtually every chromosome potentially involved. However, array-based methods have revealed a much higher frequency of submicroscopic deletions and duplications causing mental retardation (see review (Stankiewicz and Beaudet, 2007)) and autism, and a much larger fraction of autism is now believed to be caused by genetic mutations, both CNVs and point mutations, than was previously appreciated. One study of syndromic autism found genomic rearrangements in 28% of cases (Jacquemont et al., 2006) Two larger studies including both syndromic and nonsyndromic patients found de novo CNVs in 7-10% of patients (Sebat et al., 2007; marshall et al., 2008). These rearrangements are highly variable in location and size implying that mutations in many different genes, alone or in contiguous combinations, may be associated with autism. Maternally derived duplications of 15q11-q13 are the most frequently recognized genomic rearrangement in autism. A recurrent microdeletion/duplication of ˜500 kb encompassing >25 genes at chromosome region 16p11.2 has been shown to be associated with autism in ˜1% of affected individuals (Weiss et al., 2008; Sebat et al., 2007). Many additional rearrangements have been shown to be associated with isolated cases of syndromic and nonsyndromic autism but have not been reported to recur in multiple unrelated affected individuals (Sebate et al., 2007; Marshall et al., 2008). Several microduplication syndromes have now been shown to be associated with syndromic autism including duplication of the Smith-Magenis syndrome region (Potocki et al., 2007) duplication of the Williams syndrome region (Berg et al., 2007) and duplications of the Rett gene (MECP2) region in males (del Guiadio et al., 2006; Van et al., 2005). Very recently, homozygosity mapping has been used to identify autosomal recessive mutations/deletions causing autism (Morrow et al., 2008). Although deletions of 22q11.2 DGS/VCFS have been known to cause schizophrenia for many years (Murphy, 2002), there is broader emerging evidence that CNVs may also play a larger role in the etiology of psychiatric disorders such as schizophrenia (Welsh et al., 2008; Xu et al., 2008).

### BRIEF SUMMARY OF THE INVENTION

The present invention concerns identification of mutations in a particular human chromosomal locus, 15q13, associated with one or more adverse behavioral conditions, which may be considered a medical disorder to the skilled artisan. In specific embodiments, the invention concerns identification of mutations, including deletions, such as microdeletions, and/or point mutations in the CHRNA7 gene located at chromosomal locus 15q13.

In certain embodiments of the invention, there is a microdeletion at 15q13, a locus for autism, mental retardation, and psychiatric disorders with incomplete penetrance. In specific embodiments, mutation in a locus in 15q13, for example CHRNA7, will result in an individual having or being predisposed to having schizophrenia, autism, epilepsy, bipolar disorder, violent behavior, violent criminal behavior, developmental delay, seizures, mental retardation, or being a psychopath or sex offender. In some cases, the individual has not yet shown signs of the disorder, whereas in other cases the individual has shown one or more symptoms of the disorder. In particular aspects, a mutation at 15q13, for example a deletion, is substantially associated with autism, mental retardation, and psychiatric disorders, including aggressive/antisocial behavior. Behaviors such as violence, aggression, sex offenses, and drug abuse are encompassed in this embodiment.

In particular embodiments, the adverse behavioral condition to be diagnosed includes the following: violent criminal behavior, being a sex offender, psychopath, aggressive behavior, antisocial behavior, antisocial personality disorder, borderline personality disorder, conduct disorder, intermittent explosive disorder, paranoid personality disorder, paraphilia, posttraumatic stress disorder, pyromania, schizoid personality disorder, schizotypal personality disorder, substance abuse, or substance dependence. The individual may be diagnosed as having the behavioral condition or may be predisposed to having the behavioral condition.

In certain embodiments of the invention, the analysis of this locus, for example for a deletion and/or duplication, as a criminal defense and for related activities such as sentencing, predicting repeat offense, screening prior to employment or military deployment, etc. may be employed. In certain cases, screening for such a mutation may be employed in a newborn screen, or pre-symptomatic screen, including in cases where pharmaceuticals were available to reverse the phenotype. Certain embodiments of the invention include genotype analysis of the genes, sequences, miRNAs, etc. in the deleted region. In a specific embodiment, genotype analysis of any kind, including analysis of this locus, is employed as a criminal defense.

In a specific embodiment, the locus to be analyzed resides in CHRNA7, although in alternative embodiments the locus is one or more of the following: OTUD7A, KLF13, TRPM1, MTMR10, MTMR15, CHRFAM7A or FAM7A(2).

In one embodiment of the present invention, there is an isolated polynucleotide comprising a genetic marker for chromosome 15q13, wherein said genetic marker provides information for autism, mental retardation, or one or more psychiatric disorders. In a specific embodiment, the marker is in CHRNA7, OTUD7A, KLF13, TRPM1, MTMR10, MTMR15, CHRFAM7A or FAM7A(2).

In one embodiment of the invention, there is a method of identifying an individual that is predisposed to autism, mental retardation, or one or more psychiatric disorders, comprising the step of analyzing chromosome 15q13 locus from said individual.

In another embodiment of the invention, there is a method of identifying an individual that is predisposed to criminal or antisocial behavior, comprising the step of analyzing chromosome 15q13 locus from said individual. In a specific embodiment, the criminal or antisocial behavior comprises violence, aggression, sex offenses, and/or drug abuse. In a certain aspect, the individual is an infant or child. In particular embodiments of the invention, the method is further defined as including assaying for a microdeletion in the 15q13 locus.

In one embodiment, there is a method of identifying an individual that has or is predisposed to having one or more adverse behavioral conditions, comprising the step of analyzing nucleic acid from chromosome 15q13 locus from the individual, wherein the individual is or exhibits behavior associated with one or more of the following: violent criminal behavior, sex offender, psychopathy, aggressive behavior, antisocial behavior, antisocial personality disorder, borderline personality disorder, conduct disorder, intermittent explosive disorder, paranoid personality disorder, paraphilia, posttraumatic stress disorder, pyromania, schizoid personality disorder, schizotypal personality disorder, substance abuse, and substance dependence, wherein when the individual has a mutation in the locus, the individual has or is predisposed to having the adverse behavioral condition.

In a specific embodiment, methods of the invention are further defined as analyzing one or more of CHRNA7, OTUD7A, KLF13, TRPM1, MTMR10, MTMR15, CHRFAM7A or FAM7A(2).

In a particular embodiment, the individual is a sex offender or exhibits violent criminal behavior or psychopathy. In a specific embodiment, the mutation is a microdeletion or a point mutation. In specific cases, the method further comprises the step of utilizing the information from the analyzing step in a criminal proceeding under federal or state law. In some cases, the method further comprises the step of obtaining a sample from the individual. In specific embodiments, the individual is a child or an adult.

In some embodiments, the individual has already exhibited one or more of the following behaviors: aggression toward another, disregard for well-being of self or others, lack of remorse, deviant sexual behavior, uninhibited gratification in criminal, sexual, or aggressive impulses, or the inability to learn from past mistakes. In certain aspects, the individual has been convicted of one or more of homicide, rape, assault, arson, or battery. In a specific embodiment, the analyzing utilizes long-range polymerase chain reaction.

In one embodiment of the invention, there is a method of identifying an individual that is predisposed to criminal or antisocial behavior, comprising the step of analyzing chromosome 15q13 locus from the individual. In a specific embodiment, the criminal or antisocial behavior comprises violence, aggression, sex offense, and/or drug abuse. In one embodiment, the method is further defined as assaying for a microdeletion in the 15q13 locus. In certain cases, the method is further defined as analyzing one or more of CHRNA7, OTUD7A, KLF13, TRPM1, MTMR10, MTMR15, CHRFAM7A or FAM7A(2).

In certain methods of the invention, the method further comprises the step of utilizing the information from the analyzing step in a criminal proceeding under federal or state law.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### SUMMARY OF THE FIGURES

FIG. 1 shows an array-CGH analysis of 15q13 deletions. Chromosome 15-specific array CGH plots for the recurrent BP4-BP5 1.6 Mb deletion (A) and BP3-BP4 1.16 Mb deletion (B). (C) Agilent 244 k array plot for BP3-BP5 3.4 Mb deletion. (D) Physical and gene map of the region with the breakpoints involved in the recurrent and rare deletions and the position of breakpoints relative to the segmental duplication blocks (Database of Genomic Variants, http://projects.tcag.ca/variation). Regions of deletion are shown by red brackets and bars. Genes are indicted by gene symbols and breakpoint regions (BP3, BP4, and BP5) are indicated with low copy repeat blocks above each BP region.

FIG. 2 shows pedigrees for 10 families. All families have the 1.6 Mb deletion except Family 1 (1.16 Mb) and Family 2 (3.4 Mb). The patients in Families 1 and 10 are in the legal custody of a grandparent; children in Families 2, 3, and 9 are in adoptive care of unrelated families. The child in Family 8 is in foster care. Family 4 is de novo; Families 5, 6, and 7 are inherited.

FIG. 3 shows an exemplary pedigree of an affected family.

FIGS. 4A-4F show a summary of the results in patients with an isolated recurrent CHRNA7 deletion. (A) Genomic region chr15:25,800,000-31,000,000 (UCSC March 2006) with low-copy repeat clusters BP3, BP4, and BP5. The ˜680 kb deletion (B,C) and reciprocal duplication (D) is shown by the dotted vertical lines. Deletions and duplications were detected through screening the clinical lab database of array CGH studies performed in 8882 patients using oligonucleotide Chromosomal Microarray Analysis (CMA Versions 6 and 7 OLIGO) (see World Wide Web at Baylor College of Medicine Genetic Labs website). The deletions and duplications were subsequently characterized via a catalogue 2.1 M whole-genome array CGH (B) (NimbleGen Systems, Madison, Wis., USA) and a custom designed 15g13.3-specific highresolution 8×15K array CGH (C,D) (Agilent Technologies, Santa Clara, Calif., USA). BP3, BP4, and BP5 were not covered with oligonucleotide probes in the Agilent array. (E) Pedigree for patient #1 (arrow). Shaded boxes and circles indicate males and females, respectively, who have the CHRNA7 deletion. The 8-year-old propositus presented with severe mental retardation (MR) and had electroencephalographic abnormalities (abn1 EEG). The proband's mother has MR and absence seizure (SZ). The maternal aunt has MR and epilepsy. The siblings of the proband have global developmental delay (GDD). The 52-year-old maternal grandmother, who also has the deletion, has MR and was diagnosed with multiple sclerosis. (F) Schematic representation of the proposed mechanism for formation of the described CHRNA7 deletion and reciprocal duplication. Inverted chromosome region 15q13.3, present in high frequency of population, likely results from NAHR between BP4 and BP5. The second NAHR between ˜100 kb proximal (chr15:28,757,712-28,860,892) and distal (chr15:29,697,286-29,806,912) CHRNA7-LCRs (of 97.7% DNA sequence identity) on the inverted and normal region of chromosome 15q13.3, respectively, likely leads to the ˜680 kb isolated CHRNA7 deletion and reciprocal duplication. The yellow rectangle represents the ˜90 kb segment in BP4 (arrows). The analyses of the proximal breakpoint of this fragment and the distal breakpoint of the CHRNA7 deletions allowed narrowing the recombination site of the BP4-BP5 inversion to within the FAM7A1/2 genes.

FIG. 5 demonstrates exemplary missense mutations of CHRNA7.

### DETAILED DESCRIPTION OF THE INVENTION

It will be readily apparent to one skilled in the art that various embodiments and modifications may be made in the invention disclosed herein without departing from the scope and spirit of the invention.

### I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

In particular, one of skill in art recognizes that for diagnosis of psychiatric disorders one can utilize the Diagnostic and Statistical Manual of Psychiatric Disorders, Version IV Revised (DSM IV TR), which is incorporated by reference in its entirety, and from which most of the following definitions were obtained.

The term “aggressive behavior” as used herein is a term known in the art to be associated with antisocial personality disorder, intermittent explosive disorder, pyromania, borderline personality disorder, some paraphilias (involving rape, child molestation, for example), post-traumatic stress disorder, and psychopathy.

The term “antisocial behavior” as used herein refers to an individual having behavior including disregard for and violation of the rights of others. In particular cases, this is not a specific diagnostic category, though is often used interchangeably with antisocial personality disorder. It is often used in slang or common parlance to indicate a person who is introverted or not sociable, but that is an incorrect usage. This behavior generally can include irritability and aggressiveness, reckless disregard for safety of self or others, and a lack of remorse. This can be seen in antisocial personality disorder, in other psychiatric syndromes, as well as secondary to medical conditions, for example.

The term “antisocial personality disorder” as used herein refers to an individual having a pervasive pattern of disregard for and violation of the rights of others occurring at least since age 15 years, as indicated by three (or more) of the following: failure to conform to social norms with respect to lawful behaviors as indicated by repeatedly performing acts that are grounds for arrest; deceitfulness, as indicated by repeated lying, use of aliases, or conning others for personal profit or pleasure; impulsivity or failure to plan ahead; irritability and aggressiveness, as indicated by repeated physical fights or assaults; reckless disregard for safety of self or others; consistent irresponsibility, as indicated by repeated failure to sustain consistent work behavior or honor financial obligations; and lack of remorse, as indicated by being indifferent to or rationalizing having hurt, mistreated, or stolen from another. In a specific embodiment, the occurrence of antisocial behavior does not occur during the course of schizophrenia or a manic episode (in specific embodiments, a person that has schizophrenia or mania/bipolar does not have antisocial personality disorder).

The term “borderline personality disorder” as used herein refers to an individual having a pervasive pattern of instability of interpersonal relationships, self-image, and affects, and marked impulsivity beginning by early adulthood and present in a variety of contexts, as indicated by five (or more) of the following: frantic efforts to avoid real or imagined abandonment; a pattern of unstable and intense interpersonal relationships characterized by alternating between extremes of idealization and devaluation; identity disturbance; markedly and persistently unstable self-image or sense of self; impulsivity in at least two areas that are potentially self-damaging (e.g., spending, sex, substance abuse, reckless driving, binge eating); and marked reactivity of mood.

The term “conduct disorder” as used herein refer to an individual that has a repetitive and persistent pattern of behavior in which the basic rights of others or major age-appropriate societal norms or rules are violated, as manifested by the presence of three (or more) of the following: aggression to people and animals; often bullies, threatens, or intimidates others; often initiates physical fights; has used a weapon that can cause serious physical harm; has been physically cruel to people; has been physically cruel to animals; has stolen while confronting a victim (e.g., mugging, purse snatching, extortion, armed robbery); has forced someone into sexual activity; has deliberately engaged in fire setting with the intention of causing serious damage; has deliberately destroyed others' property (other than by fire); has stolen items of nontrivial value without confronting a victim (e.g., shoplifting, but without breaking and entering; forgery); is often truant from school, beginning before age 13 years; often lies; the disturbance in behavior causes clinically significant impairment in social, academic, or occupational functioning.

The term “intermittent explosive disorder” as used herein refers to an individual having several discrete episodes of failure to resist aggressive impulses that result in serious assaultive acts or destruction of property. The degree of aggressiveness expressed during the episodes is grossly out of proportion to any precipitating psychosocial stressors.

The term “paranoid personality disorder” as used herein refers to a pervasive distrust and suspiciousness of others such that their motives are interpreted as malevolent, beginning by early adulthood and present in a variety of contexts, as indicated by four (or more) of the following: suspects, without sufficient basis, that others are exploiting, harming, or deceiving him or her; is preoccupied with unjustified doubts about the loyalty or trustworthiness of friends or associates; is reluctant to confide in others because of unwarranted fear that the information will be used maliciously against him or her; reads hidden demeaning or threatening meanings into benign remarks or events; persistently bears grudges, i.e., is unforgiving of insults, injuries, or slights; perceives attacks on his or her character or reputation that are not apparent to others and is quick to react angrily or to counterattack; has recurrent suspicions, without justification, regarding fidelity of spouse or sexual partner.

The term “paraphilia” as used herein refers to recurrent, intense sexually arousing fantasies, sexual urges, or behaviors generally involving 1) nonhuman objects, 2) the suffering or humiliation of oneself or one's partner, or 3) children or other nonconsenting persons that occur over a period of at least 6 months. For some individuals, paraphilic fantasies or stimuli are obligatory for erotic arousal and are always included in sexual activity. In other cases, the paraphilic preferences occur only episodically (e.g., perhaps during periods of stress), whereas at other times the person is able to function sexually without paraphilic fantasies or stimuli. For pedophilia, voyeurism, exhibitionism, and frotteurism, the diagnosis is made if the person has acted on these urges or the urges or sexual fantasies cause marked distress or interpersonal difficulty. For sexual sadism, the diagnosis is made if the person has acted on these urges with a nonconsenting person or the urges, sexual fantasies, or behaviors cause marked distress or interpersonal difficulty. For the remaining paraphilias, the diagnosis is made if the behavior, sexual urges, or fantasies cause clinically significant distress or impairment in social, occupational, or other important areas of functioning. Paraphilic imagery may be acted out with a nonconsenting partner in a way that may be injurious to the partner (as in sexual sadism or pedophilia). The individual may be subject to arrest and incarceration. Sexual offenses against children constitute a significant proportion of all reported criminal sex acts, and individuals with exhibitionism, pedophilia, and voyeurism make up the majority of apprehended sex offenders.

The term “posttraumatic stress disorder” as used herein refers to an individual that has been exposed to a traumatic event in which both of the following were present: the person experienced, witnessed, or was confronted with an event or events that involved actual or threatened death or serious injury, or a threat to the physical integrity of self or others; the person's response involved intense fear, helplessness, or horror. While this is often thought of in a military context, it can happen post traumatically in many situations.

The term “psychopathic” or “psychopath” as used herein refers a psychological construct that describes chronic immoral and antisocial behavior. The term is often used interchangeably with sociopathy. In the International Statistical Classification of Diseases and Related Health Problems 10th Revision (ICD-10) diagnosis criteria, the terms antisocial/dissocial personality disorder are used. The term is used as a definition in law as to denote a severe condition often related to antisocial or dissocial personality disorder as defined by the Psychopathy Checklist-Revised (PCL-R). The term “psychopathy” is often confused with psychotic disorders erroneously (psychotic by denfition involves hallucinations, whereas most psychopaths do not have hallucinosis, except as a secondary condition). It is estimated that approximately one percent of the general population are psychopaths. The psychopath is defined by an uninhibited gratification in criminal, sexual, or aggressive impulses and the inability to learn from past mistakes. Individuals with this disorder gain satisfaction through their antisocial behavior and lack remorse for their actions. Lack of a conscience in conjunction with a weak ability to defer gratification and/or control aggressive desires, often leads to antisocial acts. Psychopathy has no precise equivalent in either the DSM-IV-TR, where it is most strongly correlated with the diagnosis of antisocial personality disorder, or the ICD-10, which has a partly similar condition called dissocial personality disorder.

The term “psychotic” as used herein refers to a medical description of a state of conciousness, not a diagnostic term. It can occur in many illnesses, both psychiatric (such as schizophrenia) and also in medical illnesses (such as Alzheimer's, and Parkinson's, alcohol or drug intoxication, many more). The narrowest definition of psychotic is restricted to delusions or prominent hallucinations, with the hallucinations occurring in the absence of insight into their pathological nature. A slightly less restrictive definition would also include prominent hallucinations that the individual realizes are hallucinatory experiences; however more correctly these are termed delusions. In the DSM IV, the term psychotic refers to the presence of certain symptoms. However, the specific constellation of symptoms to which the term refers varies to some extent across the diagnostic categories. In schizophrenia, schizophreniform disorder, schizoaffective disorder, and brief psychotic disorder, the term psychotic refers to delusions, any prominent hallucinations, disorganized speech, or disorganized or catatonic behavior. In psychotic disorder due to a general medical condition (i.e., in the situation where a patient has Parkinson's disease, blindness, or migraine, for example) and in substance-induced psychotic disorder (such as taking LSD), psychotic refers to delusions or only those hallucinations that are not accompanied by insight. Finally, in delusional disorder and shared psychotic disorder, psychotic is equivalent to delusional.

The term “pyromania” as used herein refers to deliberate and purposeful fire setting on more than one occasion. There is tension or affective arousal before the act, in certain cases. There often is a fascination with, interest in, curiosity about, or attraction to fire and its situational contexts (e.g., paraphernalia, uses, consequences). The individual often experiences pleasure, gratification, or relief when setting fires, or when witnessing or participating in their aftermath.

The term “schizoid personality disorder” as used herein refers to a pervasive pattern of detachment from social relationships and a restricted range of expression of emotions in interpersonal settings, beginning by early adulthood and present in a variety of contexts, as indicated by four (or more) of the following: neither desires nor enjoys close relationships, including being part of a family; almost always chooses solitary activities; has little, if any, interest in having sexual experiences with another person; takes pleasure in few, if any, activities: lacks close friends or confidants other than first-degree relatives; appears indifferent to the praise or criticism of others; shows emotional coldness, detachment, or flattened affectivity. In specific embodiments, an individual with schizoid personality disorder does not have schizophrenia or another psychotic disorder.

The term “schizotypal personality disorder” as used herein refers to a pervasive pattern of social and interpersonal deficits marked by acute discomfort with, and reduced capacity for, close relationships as well as by cognitive or perceptual distortions and eccentricities. In specific embodiments, an individual with schizotypal personality disorder does not have schizophrenia, a mood disorder with psychotic features, another psychotic disorder, or a pervasive developmental disorder.

The term “sex offender” as used herein refers to an individual that has committed a sexual act against another without that person's consent and can include but it is not necessary that the individual has been found guilty of the offense in a court of law. Sex offenders can have antisocial personality disorder, borderline personality disorder, or various paraphilias, in specific cases. As known for paraphilias, sexual offenses against children constitute a significant proportion of all reported criminal sex acts, and individuals with exhibitionism, pedophilia, and voyeurism make up the majority of apprehended sex offenders.

The term “sexual abuse of an adult” as used herein is used when the focus of clinical attention is sexual abuse of an adult (e.g., sexual coercion, rape).

The term “substance abuse” as used herein refers to a maladaptive pattern of substance use leading to clinically significant impairment or distress, as manifested by one (or more) of the following, occurring within a 12-month period: recurrent substance use resulting in a failure to fulfill major role obligations at work, school, or home (e.g., repeated absences or poor work performance related to substance use; substance-related absences, suspensions, or expulsions from school; neglect of children or household); recurrent substance use in situations in which it is physically hazardous (e.g., driving an automobile or operating a machine when impaired by substance use); recurrent substance-related legal problems (e.g., arrests for substance-related disorderly conduct); continued substance use despite having persistent or recurrent social or interpersonal problems caused or exacerbated by the effects of the substance (e.g., arguments with spouse about consequences of intoxication, physical fights).

The term “substance dependence” as used herein refers to a maladaptive pattern of substance use, leading to clinically significant impairment or distress, as manifested by three (or more) of the following, occurring at any time in the same 12-month period: tolerance, as defined by either of the following: a need for markedly increased amounts of the substance to achieve intoxication or desired effect, or markedly diminished effect with continued use of the same amount of the substance; withdrawal; the same (or a closely related) substance is taken to relieve or avoid withdrawal symptoms; the substance is often taken in larger amounts or over a longer period than was intended; there is a persistent desire or unsuccessful efforts to cut down or control substance use; a great deal of time is spent in activities necessary to obtain the substance (e.g., visiting multiple doctors or driving long distances), use the substance (e.g., chain-smoking), or recover from its effects; important social, occupational, or recreational activities are given up or reduced because of substance use; the substance use is continued despite knowledge of having a persistent or recurrent physical or psychological problem that is likely to have been caused or exacerbated by the substance.

The term “violent criminal behavior” as used herein refers to an individual that exhibits violence against at least one other individual in an act that is a crime under federal or state law. In a specific embodiment, the violent criminal behavior can include homicide, rape, assault, or battery, for example. The act may be against an adult or child, and the victim may be known to the individual or may be a stranger to the individual. In a specific embodiment, the individual has antisocial personality disorder, intermittent explosive disorder, or post-traumatic stress disorder.

### II. Certain Embodiments of the Invention

The present invention concerns diagnosis of one or more adverse behavioral conditions in an individual by assaying for mutations in one or more genes in chromosome 15q13. Symptoms of the behavioral condition may or may not have manifested at the time of the assay. The diagnosis may be performed on a child or an adult, male or female, of any individual of any race. The diagnostic information may be utilized to provide a treatment regimen for the individual; to provide information to appropriate authorities regarding the well-being of the individual and of those individuals in the surrounding environment (such as a school or workplace, for example); and/or the information may be used in a court of law in a criminal proceeding against the individual (including trial and/or sentencing of the individual), for example.

The genetic locus or loci to be assayed for the invention is present on human chromosome 15q13, and this may include identifying whether or not there is a mutation at one or more of the following genes: CHRNA7, OTUD7A, KLF13, TRPM1, MTMR10, MTMR15, CHRFAM7A or FAM7A(2). In specific embodiments, however, the gene to be assayed is CHRNA7, and part or the entire coding region and/or regulator region(s) may be assayed. In specific embodiments the mutation is a deletion, including a microdeletion, although other mutations may be identified including a point mutation, frame shift, insertion, or substitution, for example.

In particular embodiments, the adverse behavioral condition to be diagnosed includes the following: violent criminal behavior, being a sex offender, psychopathy, aggressive behavior, antisocial behavior, antisocial personality disorder, borderline personality disorder, conduct disorder, intermittent explosive disorder, paranoid personality disorder, paraphilia, posttraumatic stress disorder, pyromania, schizoid personality disorder, schizotypal personality disorder, substance abuse, or substance dependence. The individual may be diagnosed as having the behavioral condition or may be predisposed to having the behavioral condition.

### III. Detection of the Mutation

A skilled artisan recognizes that there are a variety of methods to detect a mutation in a nucleic acid sequence in addition to methods utilized herein. In specific embodiments, the mutation is detected by primer extension, polymerase chain reaction (including long-range PCR) sequencing, single stranded conformation polymorphism, mismatch oligonucleotide mutation detection, mass spectroscopy, DNA microarray, HPLC, microarray, SNP PCR genotyping, or a combination thereof, for example. In a specific embodiment of the invention, there is a fusion gene that introduces extra copies of one or more exons of CHRNA7 (such as one that introduces extra copies of exons 5-10, for example), and one can assay for this by, for example, long-range PCR to distinguish the true exons from the fusion gene exons.

Methods regarding allele-specific probes for analyzing particular nucleotide sequences are described by e.g., Saiki et al., Nature 324, 163-166 (1986); Dattagupta, EP 235,726 (U.S. Pat. No. 836,378 (Mar. 5, 1986); U.S. Pat. No. 943,006 (Dec. 29, 1986)); Saiki, WO 89/11548 (U.S. Pat. No. 197,000 (May 20, 1988); U.S. Pat. No. 347,495 (May 4, 1989)). Allele-specific probes are typically used in pairs. One member of the pair shows perfect complementarity to a wildtype allele and the other members to a variant allele. In idealized hybridization conditions to a homozygous target, such a pair shows an essentially binary response. That is, one member of the pair hybridizes and the other does not. An allele-specific primer hybridizes to a site on target DNA overlapping the particular site in question and primes amplification of an allelic form to which the primer exhibits perfect complementarily (Gibbs, 1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch impairs amplification and little, if any, amplification product is generated.

Particular nucleic acid sites can also be identified by hybridization to oligonucleotide arrays. An example is described in WO 95/11995, which includes arrays having four probe sets. A first probe set includes overlapping probes spanning a region of interest in a reference sequence. Each probe in the first probe set has an interrogation position that corresponds to a nucleotide in the reference sequence. That is, the interrogation position is aligned with the corresponding nucleotide in the reference sequence when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets are identical to the corresponding probe from the first probe set except at the interrogation position, which occurs in the same position in each of the four corresponding probes from the four probe sets, and is occupied by a different nucleotide in the four probe sets. Such an array is hybridized to a labeled target sequence, which may be the same as the reference sequence, or a variant thereof. The identity of any nucleotide of interest in the target sequence can be determined by comparing the hybridization intensities of the four probes having interrogation positions aligned with that nucleotide. The nucleotide in the target sequence is the complement of the nucleotide occupying the interrogation position of the probe with the highest hybridization intensity.

WO 95/11995 also describes subarrays that are optimized for detection of variant forms of a precharacterized nucleotide site. A subarray contains probes designed to be complementary to a second reference sequence, which can be an allelic variant of the first reference sequence. The second group of probes is designed by the same principles as above except that the probes exhibit complementarity to the second reference sequence. The inclusion of a second group can be particularly useful for analyzing short subsequences of the primary reference sequence in which multiple mutations are expected to occur within a short distance commensurate with the length of the probes (i.e., two or more mutations within 9 to 21 bases).

An additional strategy for detecting a particular nucleotide site uses an array of probes is described in EP 717,113 (U.S. Pat. No. 327,525 (Oct. 21, 1994). In this strategy, an array contains overlapping probes spanning a region of interest in a reference sequence. The array is hybridized to a labeled target sequence, which may be the same as the reference sequence or a variant thereof. If the target sequence is a variant of the reference sequence, probes overlapping the site of variation show reduced hybridization intensity relative to other probes in the array. In arrays in which the probes are arranged in an ordered fashion stepping through the reference sequence (e.g., each successive probe has one fewer 5′ base and one more 3′ base than its predecessor), the loss of hybridization intensity is manifested as a “footprint” of probes approximately centered about the point of variation between the target sequence and reference sequence.

Mundy, C. R. (U.S. Pat. No. 4,656,127), for example, discusses a method for determining the identity of the nucleotide present at a particular site that employs a specialized exonuclease-resistant nucleotide derivative. A primer complementary to the allelic sequence immediately 3′ to the site is permitted to hybridize to a target molecule obtained from a particular animal or human. If the site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the site of the target molecule was complementary to that of the nucleotide derivative used in the reaction. The Mundy method has the advantage that it does not require the determination of large amounts of extraneous sequence data. It has the disadvantages of destroying the amplified target sequences, and unmodified primer and of being extremely sensitive to the rate of polymerase incorporation of the specific exonuclease-resistant nucleotide being used.

Cohen, D. et al. (French Patent 2,650,840 (U.S. Pat. No. 4,420,902 (Dec. 20, 1983)); PCT Appln. No. WO91/02087) discuss a solution-based method for determining the identity of the nucleotide of a particular site. As in the Mundy method of U.S. Pat. No. 4,656,127, a primer is employed that is complementary to allelic sequences immediately 3′ to the site. The method determines the identity of the nucleotide of that site using labeled dideoxynucleotide derivatives, which, if complementary to the nucleotide of the site will become incorporated onto the terminus of the primer.

An alternative method, known as Genetic Bit Analysis or GBA™ is described by Goelet, P. et al. (PCT Appln. No. 92/15712 (U.S. Pat. No. 664,837 (Mar. 5, 1991); U.S. Pat. No. 775,786 (Oct. 11, 1991)). The method of Goelet, P. et al. uses mixtures of labeled terminators and a primer that is complementary to the sequence 3′ to a site in question. The labeled terminator that is incorporated is thus determined by, and complementary to, the nucleotide present in the site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase. It is thus easier to perform, and more accurate than the method discussed by Cohen.

An alternative approach, the “Oligonucleotide Ligation Assay” (“OLA”) (Landegren, U. et al., Science 241:1077-1080 (1988)) has also been described as capable of detecting a nucleotide sequence variation. The OLA protocol uses two oligonucleotides which are designed to be capable of hybridizing to abutting sequences of a single strand of a target. One of the oligonucleotides is biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Recently, several primer-guided nucleotide incorporation procedures for assaying particular sites in DNA have been described (Komher, J. S. et al., Nucl. Acids. Res. 17:7779-7784 (1989); Sokolov, B. P., Nucl. Acids Res. 18:3671 (1990); Syv anen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)).

### IV. Nucleic Acid Detection

A nucleic acid from chromosome 15q13 may be detected by any means suitable in the art. Exemplary methods and reagents related to a variety of assays regarding nucleic acid from chromosome 15q13 are described below. Although any of the nucleic acids from this chromosomal region are encompassed in this regard, CHRNA7 in a specific embodiment, and being merely exemplary in nature, is envisioned for nucleic acid detection.

a. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to CHRNA7 wildtype or mutant are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in their entirety.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). Davey et al., European Application No. 329 822 disclose a nucleic acid amplification process involving cyclically synthesizing single-stranded RNA (“ssRNA”), ssDNA, and double-stranded DNA (dsDNA), which may be used in accordance with the present invention.

Miller et al., PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

c. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al., 1989. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”), mass spectroscopy, and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNaseA mismatch cleavage assay that involves annealing single-stranded DNA or RNA test samples to an RNA probe, and subsequent treatment of the nucleic acid duplexes with RNaseA. For the detection of mismatches, the single-stranded products of the RNaseA treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from Promega Biotech. Promega markets a kit containing RNaseI that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

### V. Exemplary 15q13 Nucleic Acids for Detection

In a preferred embodiment, there is assaying of a nucleic acid sequence of 15q13 to determine whether or not an individual with the nucleic acid sequence has a mutation. In particular cases, one may assay for a mutation in the coding sequence and/or regulatory sequence of one or more of the genes. Specific genes include the following, and one of skill in art recognizes how to obtain sequences of regulatory regions when a coding sequence is provided as follows: CHRNA7 (SEQ ID NO:1); OTUD7A (SEQ ID NO:2); KLF13 (SEQ ID NO:3); TRPM1 (SEQ ID NO:4); MTMR10 (SEQ ID NO:5); MTMR15 (SEQ ID NO:6); CHRFAM7A (SEQ ID NO:7); and FAM7A(2) (SEQ ID NO:8).

Any of these genes may be assayed for one or more mutations associated with an adverse behavioral condition as described. For the sake of brevity the following passages use CHRNA7 as an example, although any of the other genes are interchangeable for the embodiments discussed below

e. Nucleic Acids and Uses Thereof

Certain aspects of the present invention concern at least one CHRNA7 wildtype and/or mutant nucleic acid. In certain aspects, the at least one CHRNA7 wildtype and/or mutant nucleic acid comprises a wild-type or mutant CHRNA7 wildtype and/or mutant nucleic acid. In certain aspects, the CHRNA7 wildtype and/or mutant nucleic acid comprises at least one transcribed nucleic acid. In particular aspects, the CHRNA7 wildtype and/or mutant nucleic acid encodes at least one CHRNA7 wildtype and/or mutant protein, polypeptide or peptide, or biologically functional equivalent thereof. In other aspects, the CHRNA7 wildtype and/or mutant nucleic acid comprises at least one nucleic acid segment of SEQ ID NO:1, or at least one biologically functional equivalent thereof.

The present invention also concerns the isolation or creation of at least one recombinant construct or at least one recombinant host cell through the application of recombinant nucleic acid technology known to those of skill in the art or as described herein. The recombinant construct or host cell may comprise at least one CHRNA7 wildtype or mutant nucleic acid, and may express at least one CHRNA7 wildtype or mutant protein, peptide or peptide, or at least one biologically functional equivalent thereof.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. In certain embodiments, the term “polymorphic” means that variation exists (i.e. two or more alleles exist) at a genetic locus in the individuals of a population. In specific embodiments “mutant” refers to a change in the sequence of a nucleic acid or its encoded protein, polypeptide or peptide that results in the individual having one or more symptoms of an adverse behavioral condition.

A nucleic acid may be made by any technique known to one of ordinary skill in the art. Non-limiting examples of synthetic nucleic acid, particularly a synthetic oligonucleotide, include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. Pat. No. 5,705,629, each incorporated herein by reference. A non-limiting example of enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells, such as recombinant DNA vector production in bacteria (see for example, Sambrook et al. 1989, incorporated herein by reference).

A nucleic acid may be purified on polyacrylamide gels, agarose, cesium chloride centrifugation gradients, or by any other means known to one of ordinary skill in the art (see for example, Sambrook et al. 1989, incorporated herein by reference).

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Thus, the present invention also encompasses at least one nucleic acid that is complementary to a CHRNA7 wildtype or mutant nucleic acid. In particular embodiments the invention encompasses at least one nucleic acid or nucleic acid segment complementary to the sequence set forth in SEQ ID NO:1. Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

One or more nucleic acid(s) may comprise, or be composed entirely of, at least one derivative or mimic of at least one nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refers to a molecule that may or may not structurally resemble a naturally occurring molecule, but functions similarly to the naturally occurring molecule. As used herein, a “moiety” generally refers to a smaller chemical or molecular component of a larger chemical or molecular structure, and is encompassed by the term “molecule.”

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and mimics. Non-limiting examples of nucleobases include purines and pyrimidines, as well as derivatives and mimics thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

Nucleobase, nucleoside and nucleotide mimics or derivatives are well known in the art, and have been described in exemplary references such as, for example, Scheit, Nucleotide Analogs (John Wiley, New York, 1980), incorporated herein by reference. “Purine” and “pyrimidine” nucleobases encompass naturally occurring purine and pyrimidine nucleobases and also derivatives and mimics thereof, including but not limited to, those purines and pyrimidines substituted by one or more of alkyl, caboxyalkyl, amino, hydroxyl, halogen (i.e. fluoro, chloro, bromo, or iodo), thiol, or alkylthiol wherein the alkyl group comprises of from about 1, about 2, about 3, about 4, about 5, to about 6 carbon atoms. Non-limiting examples of purines and pyrimidines include deazapurines, 2,6-diaminopurine, 5-fluorouracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcyosine, 5-bromouracil, 5-ethyluracil, 5-iodouracil, 5-chlorouracil, 5-propyluracil, thiouracil, 2-methyladenine, methylthioadenine, N,N-diemethyladenine, azaadenines, 8-bromoadenine, 8-hydroxyadenine, 6-hydroxyaminopurine, 6-thiopurine, 4-(6-aminohexyl/cytosine), and the like.

As used herein, “nucleoside” refers to an individual chemical unit comprising a nucleobase covalently attached to a nucleobase linker moiety. A non-limiting example of a “nucleobase linker moiety” is a sugar comprising 5-carbon atoms (a “5-carbon sugar”), including but not limited to deoxyribose, ribose or arabinose, and derivatives or mimics of 5-carbon sugars. Non-limiting examples of derivatives or mimics of 5-carbon sugars include 2′-fluoro-2′-deoxyribose or carbocyclic sugars where a carbon is substituted for the oxygen atom in the sugar ring. By way of non-limiting example, nucleosides comprising purine (i.e. A and G) or 7-deazapurine nucleobases typically covalently attach the 9 position of the purine or 7-deazapurine to the 1′-position of a 5-carbon sugar. In another non-limiting example, nucleosides comprising pyrimidine nucleobases (i.e. C, T or U) typically covalently attach the 1 position of the pyrimidine to 1′-position of a 5-carbon sugar (Kornberg and Baker, DNA Replication, 2nd Ed. (Freeman, San Francisco, 1992). However, other types of covalent attachments of a nucleobase to a nucleobase linker moiety are known in the art, and non-limiting examples are described herein.

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

A non-limiting example of a nucleic acid comprising such nucleoside or nucleotide derivatives and mimics is a “polyether nucleic acid”, described in U.S. Pat. No. 5,908,845, incorporated herein by reference, wherein one or more nucleobases are linked to chiral carbon atoms in a polyether backbone. Another example of a nucleic acid comprising nucleoside or nucleotide derivatives or mimics is a “peptide nucleic acid”, also known as a “PNA”, “peptide-based nucleic acid mimics” or “PENAMs”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. A peptide nucleic acid generally comprises at least one nucleobase and at least one nucleobase linker moiety that is either not a 5-carbon sugar and/or at least one backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

Peptide nucleic acids generally have enhanced sequence specificity, binding properties, and resistance to enzymatic degradation in comparison to molecules such as DNA and RNA (Egholm et al., Nature 1993, 365, 566; PCT/EP/01219). In addition, U.S. Pat. Nos. 5,766,855, 5,719,262, 5,714,331 and 5,736,336 describe PNAs comprising naturally and non-naturally occurring nucleobases and alkylamine side chains with further improvements in sequence specificity, solubility and binding affinity. These properties promote double or triple helix formation between a target nucleic acid and the PNA.

U.S. Pat. No. 5,641,625 describes that the binding of a PNA may to a target sequence has applications the creation of PNA probes to nucleotide sequences, modulating (i.e. enhancing or reducing) gene expression by binding of a PNA to an expressed nucleotide sequence, and cleavage of specific dsDNA molecules. In certain embodiments, nucleic acid analogues such as one or more peptide nucleic acids may be used to inhibit nucleic acid amplification, such as in PCR, to reduce false positives and discriminate between single base mutants, as described in U.S. Pat. No. 5,891,625.

U.S. Pat. No. 5,786,461 describes PNAs with amino acid side chains attached to the PNA backbone to enhance solubility. The neutrality of the PNA backbone may contribute to the thermal stability of PNA/DNA and PNA/RNA duplexes by reducing charge repulsion. The melting temperature of PNA containing duplexes, or temperature at which the strands of the duplex release into single stranded molecules, has been described as less dependent upon salt concentration.

One method for increasing amount of cellular uptake property of PNAs is to attach a lipophilic group. U.S. application Ser. No. 117,363, filed Sep. 3, 1993, describes several alkylamino functionalities and their use in the attachment of such pendant groups to oligonucleosides. U.S. application Ser. No. 07/943,516, filed Sep. 11, 1992, and its corresponding published PCT application WO 94/06815, describe other novel amine-containing compounds and their incorporation into oligonucleotides for, inter alia, the purposes of enhancing cellular uptake, increasing lipophilicity, causing greater cellular retention and increasing the distribution of the compound within the cell.

Additional non-limiting examples of nucleosides, nucleotides or nucleic acids comprising 5-carbon sugar and/or backbone moiety derivatives or mimics are well known in the art.

In certain aspect, the present invention concerns at least one nucleic acid that is an isolated nucleic acid. As used herein, the term “isolated nucleic acid” refers to at least one nucleic acid molecule that has been isolated free of, or is otherwise free of, the bulk of the total genomic and transcribed nucleic acids of one or more cells, particularly mammalian cells, and more particularly human cells. In certain embodiments, “isolated nucleic acid” refers to a nucleic acid that has been isolated free of, or is otherwise free of, bulk of cellular components and macromolecules such as lipids, proteins, small biological molecules, and the like. As different species may have a RNA or a DNA containing genome, the term “isolated nucleic acid” encompasses both the terms “isolated DNA” and “isolated RNA”. Thus, the isolated nucleic acid may comprise a RNA or DNA molecule isolated from, or otherwise free of, the bulk of total RNA, DNA or other nucleic acids of a particular species. As used herein, an isolated nucleic acid isolated from a particular species is referred to as a “species specific nucleic acid.” When designating a nucleic acid isolated from a particular species, such as human, such a type of nucleic acid may be identified by the name of the species. For example, a nucleic acid isolated from one or more humans would be an “isolated human nucleic acid”, a nucleic acid isolated from human would be an “isolated human nucleic acid”, and so forth.

Of course, more than one copy of an isolated nucleic acid may be isolated from biological material, or produced in vitro, using standard techniques that are known to those of skill in the art. In particular embodiments, the isolated nucleic acid is capable of expressing a protein, polypeptide or peptide. In other embodiments, the isolated nucleic acid comprises an isolated CHRNA7 wildtype or mutant nucleic acid sequence.

Herein certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises an CHRNA7 wildtype or mutant nucleic acid, and/or encodes an CHRNA7 wildtype or mutant polypeptide or peptide coding sequences. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the gene of interest, for example the CHRNA7 containing the A908G mutation, forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

In certain embodiments, the nucleic acid is a nucleic acid segment. As used herein, the term “nucleic acid segment”, are smaller fragments of a nucleic acid, such as for non-limiting example, those that encode only part of the CHRNA7 wildtype or mutant peptide or polypeptide sequence. Thus, a “nucleic acid segment” may comprise any part of the CHRNA7 wildtype or mutant gene sequence(s), of from about 2 nucleotides to the full length of the CHRNA7 wildtype or mutant peptide or polypeptide encoding region. In certain embodiments, the “nucleic acid segment” encompasses the full length CHRNA7 wildtype or mutant gene(s) sequence. In particular embodiments, the nucleic acid comprises any part of the SEQ ID NO:1, of from about 10 nucleotides to the full length of the sequence disclosed in SEQ ID NO:1.

A non-limiting example of the present invention would be the generation of nucleic acid segments of various lengths and sequence composition for probes and primers based on the sequences disclosed in SEQ ID NO:1, SEQ ID NO:2, SEQ ID NO:3, SEQ ID NO:4, SEQ ID NO:5, SEQ ID NO:6, SEQ ID NO:7, or SEQ ID NO:8

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The length overall length may vary considerably between nucleic acid constructs. Thus, a nucleic acid segment of almost any length may be employed, with the total length preferably being limited by the ease of preparation or use in the intended recombinant nucleic acid protocol.

In a non-limiting example, one or more nucleic acid constructs may be prepared that include a contiguous stretch of nucleotides identical to or complementary to SEQ ID NO:1. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e. all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

In particular embodiments, the invention concerns recombinant vector(s) comprising nucleic acid sequences that encode a human CHRNA7 wildtype or mutant protein, polypeptide or peptide that includes within its amino acid sequence a contiguous amino acid sequence in accordance with, or essentially as set forth in a sequence encoded by SEQ ID NO:1. In particular aspects, the recombinant vectors are DNA vectors.

In certain other embodiments, the invention concerns at least one recombinant vector that include within its sequence a nucleic acid sequence essentially as set forth in SEQ ID NO:1. In particular embodiments, the recombinant vector comprises DNA sequences that encode protein(s), polypeptide(s) or peptide(s) exhibiting CHRNA7 wildtype or mutant activity.

The term “functionally equivalent codon” is used herein to refer to codons that encode the same amino acid, such as the six codons for arginine and serine, and also refers to codons that encode biologically equivalent amino acids, which are well known in the art.

Information on codon usage in a variety of non-human organisms is known in the art (see for example, Bennetzen and Hall, 1982; Ikemura, 1981a, 1981b, 1982; Grantham et al., 1980, 1981; Wada et al., 1990; each of these references are incorporated herein by reference in their entirety). Thus, it is contemplated that codon usage may be optimized for other animals, as well as other organisms such as fungi, plants, prokaryotes, virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1 will be nucleic acid sequences that are “essentially as set forth in SEQ ID NO:1”.

It will also be understood that this invention is not limited to the particular nucleic acid sequences of SEQ ID NO:1, respectively. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have mutant amino acids sequences.

The nucleic acids of the present invention encompass biologically functional equivalent CHRNA7 wildtype or mutant proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine CHRNA7 wildtype or mutant protein, polypeptide or peptide activity at the molecular level.

Fusion proteins, polypeptides or peptides may be prepared, e.g., where the CHRNA7 wildtype or mutant coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

As used herein an “organism” may be a prokaryote, eukaryote, virus and the like. As used herein the term “sequence” encompasses both the terms “nucleic acid” and “proteinaceous” or “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e. an “enzymatically produced” sequence) or biological production in vivo (i.e. a “biologically produced” sequence).

### VI. Kits of the Invention

In certain embodiments of the invention, there is a kit comprising one or more reagents for the detection of a mutation in CHRNA7, including, for example one or more primers for CHRNA7, polymerase, polynucleotides, buffers, sequencing reagents, etc.

All of the useful materials and/or reagents required for detecting CHRNA7 sequences (mutant or wildtype (for example as a control)) in a sample may be assembled together in a kit. This generally will comprise a probe or primers designed to be suitable for detection of the mutation. In an alternative embodiment, the probe or primers are designed to hybridize specifically to individual nucleic acids of interest in the practice of the present invention, including CHRNA7 sequences. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

Any of the compositions described herein for identification of a mutation in CHRNA7 may be comprised in a kit. In a non-limiting example, a primer, control nucleic acid (including wildtype CHRNA7), and amplification reagents may be comprised in a kit in suitable container means. The components of the kits may be packaged either in aqueous media or in lyophilized form, for example. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed, in certain cases. However, various combinations of components may be comprised in a vial. The kits of the present invention also will typically include a means for containing the components of the kit in close confinement for commercial sale. Such containers may include injection or blow molded plastic containers into which the desired component containers are retained, for example.

### EXAMPLES

The following examples are offered by way of example and are not intended to limit the scope of the invention in any manner.

### Example 1

### Microdeletion 15Q13: A Locus for Autism, Mental Retardation, and Psychiatric Disorders with Incomplete Penetrance

Microdeletions within chromosome 15q13 are associated both with a newly recognized syndrome of mental retardation, seizures, and dysmorphic features and with schizophrenia. The inventor identified 14 individuals (11 children and 3 parents in 10 families) with microdeletions of 15q13. Phenotypes in the children included developmental delay or mental retardation (9/11), autistic spectrum disorder (at least 4-5/11), speech delay, aggressiveness, attention deficit hyperactivity disorder, and other behavioral problems. Both parents were available in 4 families, and the deletion was de novo in one, inherited from an apparently normal mother in two, and inherited from a father with learning disability and bipolar disorder in a fourth family. Of the 11 children, 7 in 6 families were not living with their biological parents, and DNA was available for only 1 of these 12 parents; the deletion was presumably inherited for one of these families with two affected children. Among the unavailable parents, two mothers were described as having mental retardation, another mother as having “mental illness,” and one father as having schizophrenia. In certain embodiments, some of the unavailable parents have the deletion. Thus, in addition to mental retardation and schizophrenia, deletion 15q13 is strongly associated with autism and perhaps with other psychiatric disorders, but it also can occur with a normal phenotype. A high rate of adoption may be caused by the presence of the deletion in biological parents. In certain embodiments, histories of antisocial behaviors in unavailable biological parents indicate that deletion 15q13 is associated with such behaviors.

The recurrent 15q13 microdeletion syndrome resulting in loss of a ˜1.6 Mb segment (3.95 Mb in one case) was first described using whole genome array comparative genomic hybridization (aCGH) and quantitative PCR screening of individuals with mental retardation and/or congenital anomalies (Sharp et al., 2008). This genomic region is immediately distal to the common 5-6 Mb 15q11-q13 deletion resulting in Prader-Willi syndrome (PWS) or Angelman syndrome (AS). The presence of segmental duplication blocks of sufficient size and orientation predict that this recurrent deletion is most likely mediated by NAHR between these segmental duplications (Sharp et al., 2008; Sharp et al., 2005). These segmental duplications are involved in previously reported breakpoints (BP3, BP4, and BP5) that are associated with atypical larger deletions resulting in AS or PWS and with inv dup 15q (Stankiewicz and Lupski, 2002; Sahoo et al., 2006; Sahoo et al., 2007; Wang et al., 2004; Sharp et al., 2006). The first report of 15q13 deletions including Bp4-BP5 found that two cases were de novo and three others inherited from an affected parent. The 15q13 microdeletion was not found in a cohort of 960 normal individuals or in 2002 control individuals that were studied for genome-wide copy number variations (Sharp et al., 2008). Mental retardation and seizures were present in most of the individuals indicating dosage sensitivity for one or more genes within the deleted interval. The high frequency of seizures among the patients was suggested to be due to haploinsufficiency for the α7 nicotinic acetylcholine receptor gene (CHRNA7) within the deleted region. Testing of large cohorts of patients with mental retardation detected a prevalence of about 0.3% among individuals with mental retardation. Based on the NAHR mechanism, it was anticipated that the complementary duplication of the same genomic segment might occur; indeed two control individuals with such a duplication were identified (Sharp et al., 2008). Two recent reports found deletions of 15q13 as one of the most common identifiable genetic causes of schizophrenia (Stone et al., 2008; Steafansson et al., 2008).

The Medical Genetics Laboratories (MGL) at Baylor College of Medicine (BCM) have performed aCGH on >14,000 cases referred between February 2004 to May 2008; the most common reasons for testing are developmental delay, mental retardation, dysmorphic features, congenital anomalies, autism, and general suspicion of a chromosomal anomaly (Lu et al., 2007). These samples were analyzed on consecutive versions of targeted arrays, initially BAC-based and later oligonucleotide-based as described previously (Lu et al., 2007; Ou et al., 2008). Coverage for the 15q13 BP3-BP4 region was present from very early in the series, but a BAC for the more important BP4-BP5 region was not introduced until the first 5800 samples had been analyzed. The first deletion was detected in December 2006, and a total of 14 independent families (a 15^{th} was found in a sample from the South Carolina Autism Project) deleted for 15q13 were detected for frequency of ˜0.17% [14/(14,000-5800)] in samples submitted to the diagnostic laboratory. Three of the cases included here were tabulated as V5-77, V5-80, and V5-81 in the supplemental materials of an earlier publication (Lu et al., 2007). All patients were of European descent (white) except that one father of an adopted child was African-American. For further analysis, a chromosome 15-specific oligonucleotide array was utilized, having extensive coverage across the 15q11.2q14 region including the AS/PWS critical interval to better define the boundaries of the deletions (Sahoo et al., 2008). The present invention provides genotype and phenotype data for 14 individuals (11 children and three parents) from 10 families with copy number loss in the 15q13 region. Initial identification of the cases by aCGH was based on loss of a genomic segment interrogated by BAC clones or by oligonucleotides within the BACs RP11-348B17 only (chr15:29,067,933-29,289,407) in 8/10 families, by BAC clone RP11-143J24 only (chr15:27,906,198-28,091,821) in 1/10 families, and for both BACs in 1/10. The deletions were confirmed in all of the cases by fluorescence in situ hybridization (FISH).

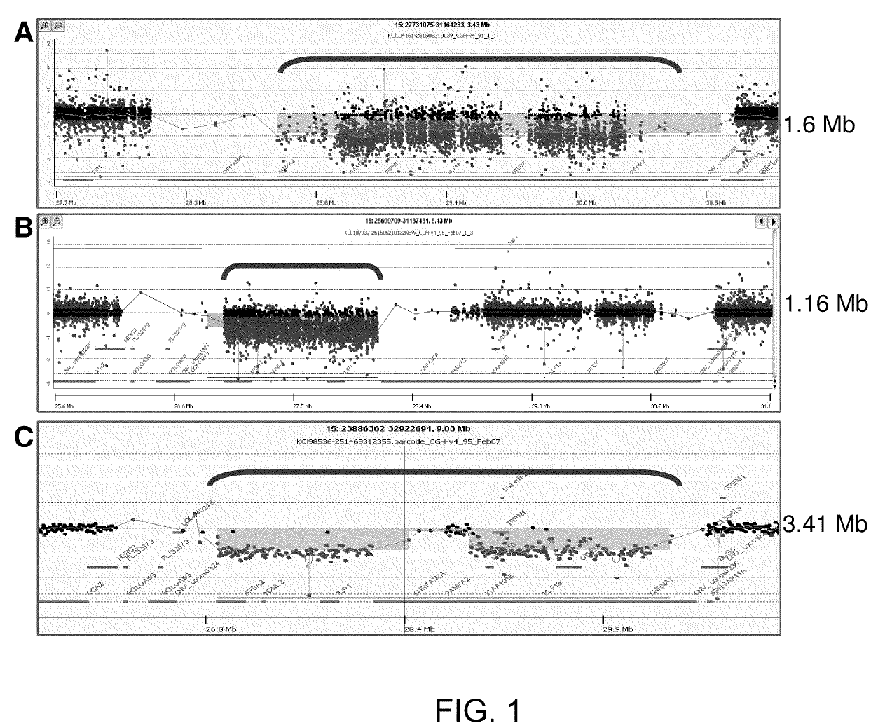

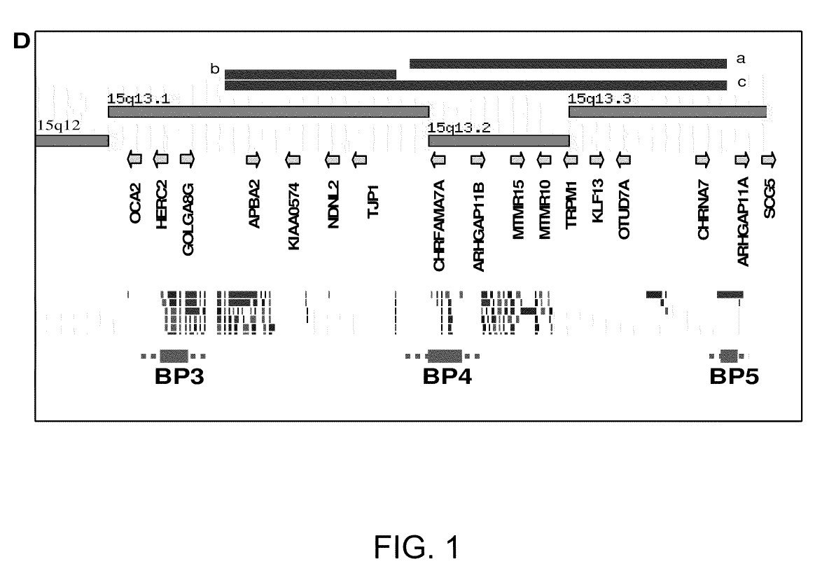

Examples of the detection of the deletions are shown in FIG. 1A-C using the custom-designed chromosome 15-specific oligonucleotide array (Sahoo et al., 2008) or using a commercial whole-genome 244 k array (both from Agilent Technologies, Santa Clara, Calif.). The most common BP4-BP5 deletion of 1.6 Mb corresponds to 15q13.2q13.3 and was seen in 8/10 families (FIG. 1A), while the smallest BP3-BP4 deletion of 1.16 Mb corresponds to 15q13.1q13.2 (FIG. 1B) and was present in one family. The largest BP3-BP5 deletion of 3.4 Mb corresponds to 15q13.1q13.3 (FIG. 1C) and was seen in one family. The genomic architecture of this region is shown in FIG. 1D.

Furthermore, as predicted by the NAHR mechanism, we detected four cases of duplication of the BP4-BP5 segment; it is not clear at present whether duplications in this region are benign or may cause phenotypic abnormalities. Based on their size and breakpoints, these duplications likely represent the reciprocal recombination product to the ˜1.6 Mb deletions.

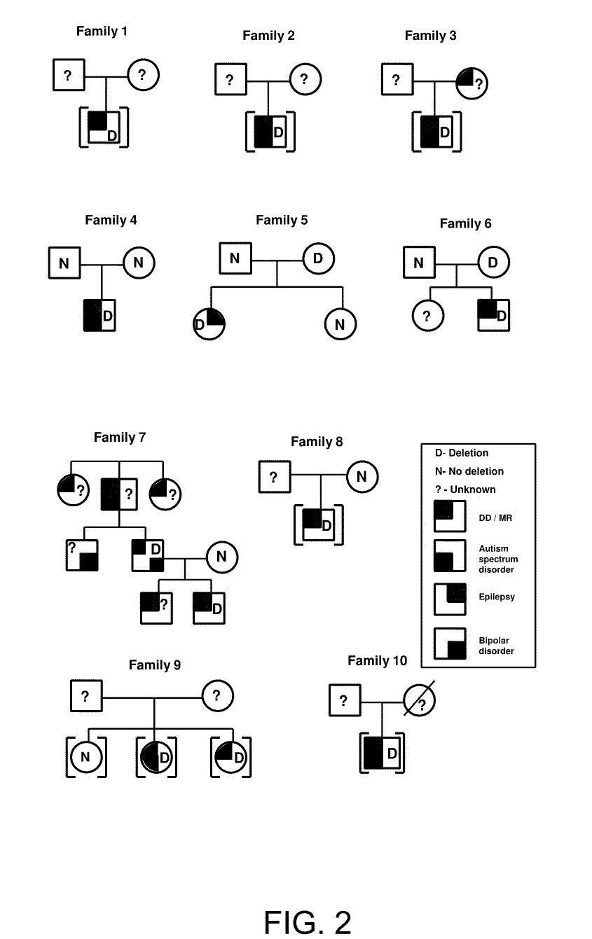

The clinical descriptions of the families reported here are without photographs for reasons of confidentiality as explained below. Similar to the cases described previously, developmental delay or mental retardation was detected in 9/11 of the children with low normal IQ in the other two (Table 1 and FIG. 2) and all 11 had at least mild mixed expressive and receptive language delay. Importantly, at least 4-5/11 of the children detected with 15q13 microdeletion had symptoms within the range of autism spectrum disorder (ASD), one of them being diagnosed with Asperger syndrome. Interestingly, only one of the two siblings in Family 9 had ASD based on thorough evaluation in an autism research center, implying variable expressivity of this behavioral phenotype among the patients. Autism was not frequent in the first report of 15q13 deletions with only one case with “mild autism” detected (Sharp et al., 2008). Notably, even among the seven probands described herein who did not have ASD, most were not tested with autism-specific diagnostic instruments, and language impairment was prominent and more severe than the developmental delay in the gross and fine motor skills, suggesting that additional patients in this report might meet criteria for autism if properly tested and that there is a disproportionate effect of 15q13 deletion on language skills and communication. Abnormal behaviors including aggressiveness, repeated head banging, and/or attention deficit hyperactivity disorder (ADHD) were detected in 5/11 (Table 1) of the patients. Although aggressive behavior was not frequent in the previous report of 15q13 deletions, one boy with ˜1.6 Mb deletion and “mild autism” was hospitalized five times in psychiatric facilities due to aggression and rage.

The facial appearances were generally consistent with those already published. The appearance is variable and ranged from near normal to moderately dysmorphic. Common facial features in our patients as well as in the previously described patients include hypertelorism, short philtrum, and everted and thick upper lip. At least two of our patients were thought initially to have coarse facies that prompted further testing for disorders associated with facial coarseness, including mucopolysaccharidoses and Coffin-Lowry syndrome. Mild digital aberrations, including brachydactyly and clinodactyly were observed in five of our patients, similar to the previously described cases (Sharp et al., 2008).

Epilepsy was observed in only two of the 11 children and in none of the three parents with the deletion. Another child had convulsive like episodes not associated with abnormal electrical encephalographic activity. The prevalence of seizures therefore is significantly lower in our cohort than in the initial report of this syndrome. This difference was observed regardless of the fact that 9/10 of our families had deletion of CHRNA7, a gene that was postulated to mediate the epilepsy in 15q13 deletions.

Both parents were available for deletion testing in only four families, and analysis of the mother alone was possible in a fifth. The deletion was confirmed to be a de novo event in a single family (F 4). The deletion was inherited in three of the four families (F5, F6, F7) where both parents available for analyses, and presumptively inherited in a family (F9) with affected siblings but parental samples not available, making the deletion inherited in four of five families where determinable. In one family (F 7), the deletion was inherited from the father who had learning disabilities by self report and was diagnosed with bipolar disorder. In two families (F5 & F6), the deletion was inherited from the mother, and these women were perceived by themselves, family members, and physicians as being normal based on education, employment, and rearing of a family, and they did not have a history of cognitive impairment or convulsions, although formal assessments of encephalography, cognition and behavior were not performed. Dysmorphic features in these three parents were either entirely absent or extremely subtle. Some clinically normal siblings were negative for the deletion as shown in FIG. 2. Deletion carriers with a normal phenotype were not observed for the BP4-BP5 deletion in the previous report, although a BP3-BP4 deletion was found in a normal father (Sharp et al., 2008).

It was not possible to draw any conclusions regarding genotype-phenotype correlations between deletion size and phenotype, because only two cases had other than the common 1.6 Mb deletion. The wide range and heterogeneity of phenotypic expression for the common 1.6 Mb deletion is noteworthy and includes normal phenotype, mental retardation, autism, and schizophrenia based on the families reported here and on publications of others. Combining the present cases with those reported previously, only 4 out of the 17 families with 15q13 deletion syndrome did not have the common 1.6 Mb deletion; two with a larger ˜3.4-3.9 Mb deletion located between BP3 and BP5, and two with ˜1.16 Mb deletion located between BP3 to BP4 (FIG. 1). The phenotype for the 1.6 Mb deletion can include individuals who function normally in society, although subtle cognitive deficits or behavioral susceptibilities have not been ruled out in these individuals. Also the mental retardation in the affected children was usually not severe, and may often allow living independently, particularly if many of the unavailable biological parents in the study prove to have the deletion. Based on the observation of only two families with the BP3-BP4 deletion, one of which included a normal father with the deletion, the phenotypic significance of the BP3-BP4 deletion is unclear.

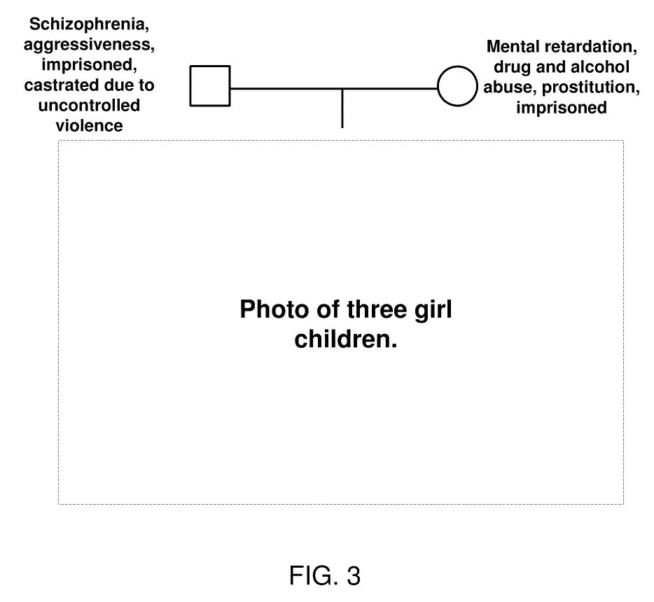

The most remarkable aspect of this report is that 64% (7 of 11) of the children were in adoptive care; five children were adopted in infancy though and two were legally adopted by or in the legal custody of grandparents. None of the parents were deceased at the time of adoption. This indicates that the adoption in most or all cases was related to cognitive, psychiatric, and/or social issues in the biological parents. For comparison, in the general population of the United States about 2.5% of the children are adopted (see census website on the World Wide Web), although a precise rate of adoption or custody by other than the biological parents is not known for children with disabilities, and the rate is probably higher than for normal children, because of both acquired or inherited disabilities in the biological parents. In some embodiments, some of the unavailable biological parents have the deletion, and in specific embodiments one or the other parent presumably has the deletion in the case of the affected siblings (F9). In certain aspects, the phenotype caused by the deletion in a parent contributed to the outcome of adoption, but also deletions of BP4-BP5 are frequently inherited rather than de novo, 4/5 as described herein and 2/4 as described previously (Sharp et al., 2008). Although mental retardation was reported for two unavailable biological mothers, “mental illness” for another mother, and schizophrenia for one biological father, there is no direct documentation of these diagnoses. The reports regarding the biological parents raise the possibility that the deletion could be inherited in a maximum of 9 of the 10 families reported here. There were unconfirmed reports of antisocial behavior for numerous biological parents (see FIG. 3 and Table 2). In specific embodiments, deletion at 15q13 is associated with antisocial behaviors.

As smaller submicroscopic deletions and duplications are identified, it is likely that phenotypes are milder, that penetrance is less than 100%, and that a larger fraction of cases are inherited. In certain aspects of the invention, duplications will confer milder phenotypes with lower penetrance as seems to be the case for duplications of the DGS/VCFS, WBS, and SMS regions.

There is now very substantial evidence that the BP4-BP5 deletion of 1.6 Mb causes a mental retardation phenotype with high but not complete penetrance and can also cause schizophrenia. The genomic structure for this region is poorly defined in even recent assemblies of the genome; the region is highly complex and repetitive and likely highly variable among chromosomes. A recent publication goes a substantial way towards delineating the region (Makoff and Flomen, 2007). This interval contains six well-characterized genes; CHRNA7, OTUD7A, KLF13, TRPM1, MTMR10, and MTMR15, while CHRFAM7A and FAM7A(2) may be imbedded in BP4 and BP5 (FIG. 1d). The ARHGAP11B and ARHGAP11A genes appear to be imbedded in BP4 and BP5, respectively. The CHRNA7 gene encodes the α7 subunit of the neuronal nicotinic receptor, which is a homopentameric synaptic ion channel protein [MIM 118511]. Although there are reports of genetic linkage to this region for juvenile myoclonic epilepsy (Elmslie et al., 1997) and for rolandic epilepsy (Neubauer et al., 1998), these results do not implicate the CHRNA7 gene specifically any more that the immediate neighboring genes. Mice heterozygous or homozygous for a null mutation in Chrna7 were phenotypically normal, although neither social behavioral tests nor induction of seizures were performed (Orr-Urtreger et al., 1997), and one group has reported a minor impairment in the delayed matching-to-place task (Fernandes et al., 2006). There are numerous publications suggesting a possible role for the CHRNA7 gene in schizophrenia as reviewed elsewhere (Flomen et al., 2006; Martin and Freedman, 2007). Some of these reports suggest linkage or linkage disequlibrium, and implicate the BP4-BP5 region perhaps but not necessarily CHRNA7 specifically (Freedman et al., 1997; Freedman et al., 2001), while some speculation involves the biology of the α7 nicotinic receptor and auditory gating. The CHRNA7 gene has not been implicated specifically in autism except for reports of loss of α7-immunoreactive neurons in certain regions of autism brain (Ray et al., 2005). There is a CHRFAM7A fusion gene embedded in BP4 with an additional copy possibly imbedded in BP5 on some chromosomes, but the function of this gene, if any, is unclear. The fusion gene includes exons 5-10 of CHRNA7 fused to five exons of gene family member 7A (FAM7A). (Gault et al., 1998) FAM7A(2) appears to be imbedded in both BP4 and BP5 (Makoff and Flomen, 2007). There is an inversion of CHRFAM7A in the human population, and both orientations occur with similar frequency (Flomen et al., 2008). There is a CNV involving CHRFAM7A, and there is a recent report of association between this CNV and the major psychoses (Flomen et al., 2006). The possibility of NAHR between the homologous stretches of these two genes as a mechanism for a deletion CNV perhaps resulting in a susceptibility locus for neurological traits also should be considered.

The other genes in the BP4-BP5 region should not be neglected as candidates to cause a neurologic phenotype by virtue of an excessively narrow focus on CHRNA7. The OTUD7A gene encodes a deubiquitinase that is thought to be a negative regulator of innate immune responses (Kayagaki et al., 2007), but a role in the brain is possible. KLF13 belongs to a family of transcription factors that contain three classical zinc fingers (Outram et al., 2008), KLF13 homozygous null mice show splenomegaly and modified erythropoiesis, but no obvious neurological phenotype (Gordon et al., 2008). The TRPM1 gene encodes a protein that is a member of the transient receptor potential (TRP) cation channel family of proteins, and there is evidence for limited expression of TRPM1 in mouse brain (Kunert-Keil et al., 2006) (Allen Brain Atlas database on the World Wide Web). MTMR10 and MTMR15 are members of a family of myotubularin myopathy-related genes which encode phosphoinositide-metabolizing phosphatases (Tronchere et al., 2003).

There is less information available but three cases (one reported here and two previously (Xu et al., 2008; Sharp et al., 2008) leave substantial doubt as to whether deletion from BP3 to BP4 causes mental retardation or not. In both cases where parents were tested, a normal parent carried the deletion, but the patient was in the care of a grandparent in our case, and samples from the parents are not available. There are four genes located between BP3 and BP4: TJP1, NDNL2, KIAA, and APBA2, and all are deleted in our case with the small 1.16 Mb deletion (BP3-BP4) and in the case with the large 3.4 Mb deletion located at BP3-BP5 (families F1 and F2, respectively in Table1). Based on the data described herein, the existence of the deletion in two unaffected parents may represent incomplete penetrance of the phenotype and does not exclude pathogenic effects of the deletion. Moreover, the fact that the deletion has not been detected among controls (Sharp et al., 2008) leaves open the possibility that this deletion has pathological potential with incomplete penetrance rather than representing a benign polymorphic CNV. A de novo duplication of these genes was detected recently in a patient with schizophrenia (Kirov et al., 2008). One of these genes, APBA2 encodes amyloid precursor-binding protein A2 that directly interacts with neurexins (Biederer and Sudhof, 2000). Interestingly neurexins belong to a family of presynaptic proteins that interact with the neuroligins, their post synaptic partners; two of its members are associated with neurological disorders, including autism (Biederer and Sudhof, 2000; Kim et al., 2008) and schizophrenia (Murphy, 2002). Mice lacking APBA2 (Mint2), together with the other brain specific Mint isoform-Mint1, have a decline in spontaneous neurotransmitter release, lowered synaptic strength, and enhanced paired-pulse facilitation, suggesting abnormalities of presynaptic neurotransmitter release (Ho et al., 2006). Although the deletion among the great majority of our patients did not include APBA2, one cannot exclude a position effect of the downstream deletion on APBA2 expression and function. Tight junction protein 1 (TJP1), also known as zonula occludens protein 1 (ZO-1), is located on a cytoplasmic membrane surface of vertebrate intercellular tight junctions. The necdin-like 2 (NDNL2) gene is a little studied member of the MAGE superfamily. Most likely haploinsufficiency for individual genes or combinations of genes within BP4-BP5 cause the mental retardation phenotype associated with that deletion phenotype, although position effects between the two regions or involving flanking regions cannot be ruled out.

Overall, the recurrent 15q13 deletions comprise a recently recognized microdeletion syndrome that follows a classical NAHR based mechanism. In addition to the reported occurrence with mental retardation, seizures, dysmorphisms, and schizophrenia, a diagnosis of autism is common, and the inventor has observed lack of penetrance, and a parent with the deletion and a diagnosis of bipolar disorder. This extremely broad phenotypic spectrum with the BP4-BP5 deletion is remarkable. The deletion is more frequently inherited rather than de novo; affected children are often in the care of other than the biological parents; and unconfirmed reports of antisocial behavior in biological parents are of concern. In addition, this deletion is an example of a genomic disorder associated with both normal and abnormal phenotype and demonstrates that the presence of a CNV in a normal parent does not exclude phenotypic abnormality in a child, or in the prenatal context, in a fetus.

TABLE 1
Certain Individuals with 15q13 deletions

| | Age | | Language | | | | |
|---|---|---|---|---|---|---|---|
| | yr. | DD or MR/ | delay/ | | | | |
| Family | gender | autism | seizures | Wgt/Hgt/FOC | Behavior | Other | Inheritance |
| F1-1 | 5 M | Moderate/ | Moderate/no | >>97%/>90%/ | None reported | Digital findings | In custody of maternal |
| | | no | | >>97% | | | grand mother; maternal |
| | | | | | | | great-grandfather with a |
| | | | | | | | diagnosis of |
| | | | | | | | schizophrenia |
| F2-1 | 3 M | DQ 58/ | Moderate/no | 50%/30%/ | Mild sterotypic | Benign | Adopted to unrelated |
| | | Autism | | >>97% | | hydrocephalus | family; little |
| | | disorder | | | | mild Chiari I | information on |
| | | | | | | | biological parents. |
| F3-1 | 7 M | IQ 82/ | Mild/no | 95%/>97%/ | ADHD | | Adopted to unrelated |
| | | Asperger. | | 90% | | | family; mother said to |
| | | | | | | | have mental retardation. |
| F4-1 | 2 M | Moderate/ | Severe/no | All 50-75% | None reported | Normal MRI | De Novo |
| | | borderline | | | | | |
| F5-1 | 13 F | Normal or | Mild/yes | 65%/7%/? | None reported | Normal MRI | Mother has the deletion. |
| | | mild/no | | | | | |
| F5-3 | Ad F | Normal/no | No/no | Normal | Normal | | Mother of F5-1; normal |
| Mo 5-1 | | | | | | | phenotype. |
| F6-1 | 4 M | Moderate/ | Moderate/no | 44%/11%/75% | None reported | Digital findings, | Mother has the deletion. |
| | | no | | | | arachnoid cyst | |
| F6-3 | Ad F | Normal/no | no/no | Normal | Normal | | Mother of F6-1; normal |
| Mo 6-1 | | | | | | | phenotype. |
| F7-1 | 3 M | Mild/no | Moderate/no | 92%/>89%/ | Aggressive, | | Father has deletion, |
| | | | | 50% | self-injurious | | learning disabilities, |
| | | | | | | | bipolar disorder. |
| F7-2 | Ad M | Learning | Likely/no | Normal | None reported | | Father of F8-2; learning |
| Fa 7-2 | | disabilities/ | | | | | disabilities and bipolar |
| | | no | | | | | disorder |
| F8-1 | 3 F | Moderate/ | Moderate/no | 25%/50%/50% | Head banging | Digital findings, | Mother learning |
| | | no | | | | MRI normal | disorder but no deletion. |
| | | | | | | | In foster care. Removed |
| | | | | | | | from parental care 15 mo |
| | | | | | | | age. |
| F9-1 | 12 F | Moderate/ | Moderate/no | Normal range | Poor sleep | Long digits, | Three sibs adopted to |
| | | yes? | | | | hyperextensible | same unrelated family; |
| | | | | | | joints; mild | 2 affected and 1 |
| | | | | | | facial | unaffected sib; mother |
| | | | | | | dysmorphism | mental retardation; |
| | | | | | | | father schizophrenia |
| F9-2 | 9 F | Moderate/ | Moderate/no | Normal range | ADHD | Long digits, | Sibling of F9-1 |
| | | no | | | | hyperextensible | |
| | | | | | | joints; facial | |
| | | | | | | dysmorphism | |
| F10-1 | 10 M | IQ 27/ | Severe/ | 50%/75% (10 y)/ | Extremely | Mild facial | In custody of paternal |
| | | autism | autism | 50% birth | hyperkinetic | dysmorphism | grand mother?; mother |
| | | disorder | disorder | | and very | | deceased and had |
| | | | | | aggressive | | “mental illness.” Father |
| | | | | | behaviors | | normal. |

TABLE 2
Biological parents of children in adoptive care

| Family | Fathers | Mothers |
|---|---|---|
| 1 | No history | Drug/alcohol abuse, prison |
| 2 | No history | No history |
| 3 | No history | Mental retardation, epilepsy, |
| | | depression, prostitution, prison |
| 8 | Sex offender listed in two | Learning disorder and |
| | states, prison | multiple sclerosis, deletion |
| | | negative |
| 9 | Schizophrenia, | Mental retardation, |
| | aggressiveness, uncontrolled | drug/alcohol abuse, |
| | violence, sex offender | prostitution, prison |
| | castrated, prison | |
| 10 | Normal | Mental illness, drug/alcohol |
| | | abuse, deceased |

### Example 2

### Isolated Recurrent Deletions Of CHRNA7 Associated with a Wide Range of Neurodevelopmental Phenotypes

There is an isolated recurrent ˜680 kb deletion of CHRNA7 in ten individuals from four unrelated families with neurodevelopmental phenotypes, including developmental delay, mental retardation, and seizures. The deletion cosegregates with the phenotype in one family with six affected individuals. This Example shows that this deletion and its reciprocal duplication result from nonallelic homologous recombination between low-copy repeats on the normal and inverted region of chromosome 15q13.3, in particular aspects of the invention. The results, in combination with previous genotype/phenotype data for deletion 15q13.3, indicate that haploinsufficiency of CHRNA7 is causative for the majority of phenotypes in the recently described microdeletion 15q13.3 syndrome.

Recurrent deletions of chromosome 15q13.3 were not recognized as pathological until 2008, when they were first reported in patients with mental retardation, seizures, autism, and bipolar disorder (Sharp et al., 2008; Miller et al., 2009; Ben-Shachar et al., 2009; van Bon et al., 2009; Stefansson et al., 2008; International Schizohrenia Consortium, 2008). This deletion was also found in ˜1% of individuals with idiopathic generalized epilepsy (Helbig et al., 2009). In contrast to many analogous deletion syndromes, which occur as de novo events, the affected subjects reported to date typically inherit the mutation from a parent, who may have any of the known phenotypic manifestations or occasionally may have a normal phenotype. The reciprocal duplication 15q13.3 also was reported at a lower frequency; however, currently, there is no clear evidence as to whether it is invariably benign or may cause phenotypic abnormalities with a milder phenotype and/or lower penetrance.

The critical 1.5 Mb deletion 15q13.3 likely arises through nonallelic homologous recombination (NAHR) between low copy repeat (LCR) sequences designated as breakpoints 4 and 5 (BP4 and BP5) telomeric to the greater Prader-Willi/Angelman syndrome domain. This chromosomal region encompasses six RefSeq genes: MTMR15, MTMR10, TRPM1, KLF13, OTUD7A, and CHRNA7. Attention has focused on CHRNA7 because of its association with epilepsy (Taske et al., 2002) and its possible implication in schizophrenia (Leonard and Freedman, 2006; Stephens et al., 2009). CHRNA7 encodes the α7 subunit of the neuronal nicotinic acetylcholine receptor, which is highly expressed in the brain. A fusion gene CHRFAM7A mapping in BP4 is comprised of the exons A-E of FAM7A1/2 and exons 5-10 of CHRNA7. Haplotype analysis is likely to be flawed for the SNPs currently shown in genome assemblies to occur in both CHRNA7 and the CHRFAM7A fusion gene. Any attempts at sequencing to detect mutations must distinguish the sequences for exons 5-10 from CHRNA7 and CHRFAM7A.

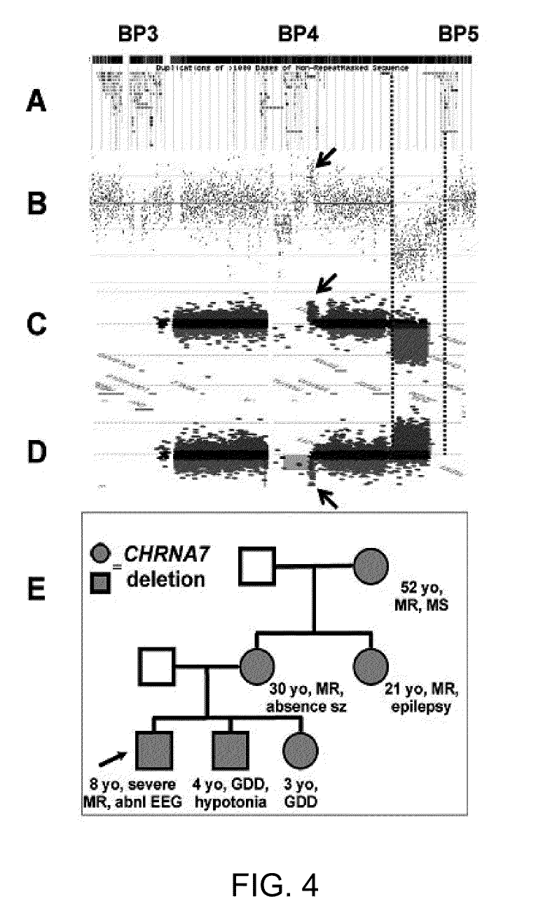

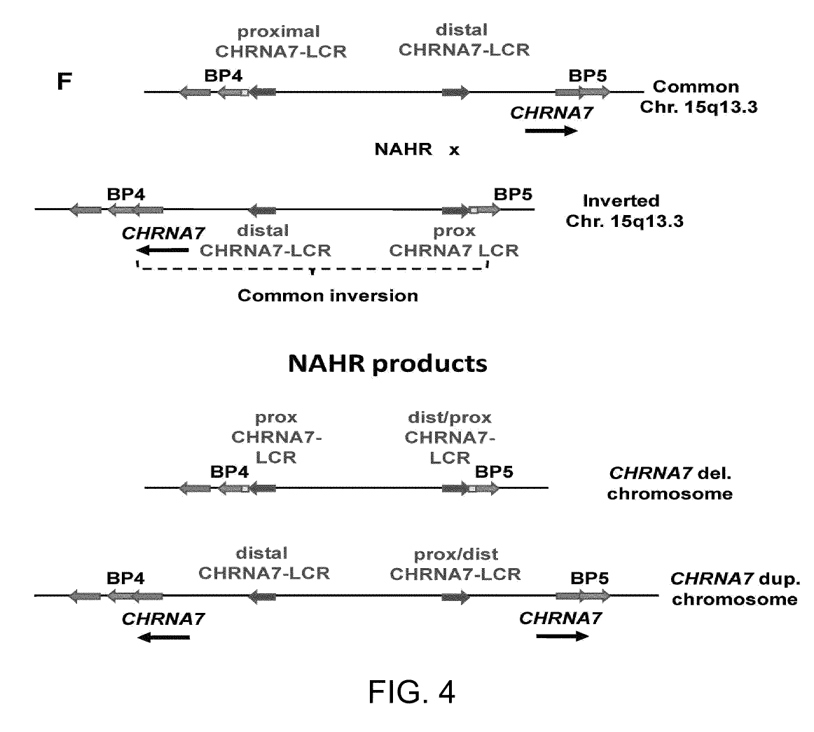

There are ten individuals from four unrelated families with a smaller ˜680 kb deletion that lies within the 1.5 Mb deletion 15q13.3 and encompasses only the entire CHRNA7 gene and the first exon of one of the predicted isoforms of OTUD7A (FIG. 4A-4D). The reciprocal duplication is far more common than the deletion and potentially leads to over-expression of solely CHRNA7. Although an exon of OTUD7A is included, hereafter these genotypes are referred to as deletion CHRNA7 and duplication CHRNA7. The patients with deletion CHRNA7 presented with various combinations of phenotypes falling within the range of those seen for deletion 15q13.3 syndrome (Table 3).

TABLE 3
Phenotypic features of individuals with CHRNA7 deletion

| | | | | | Patient 5 | Patient 6 | Patient 7 | | Patient 9 | Patient 10 |
|---|---|---|---|---|---|---|---|---|---|---|
| | | | | | (sibling | (sibling | (mother | Patient 8 | (grand- | (mother |
| | | | | | of | of | of | (aunt of | mother | of |
| | Patient 1 | Patient 2 | Patient 3 | Patient 4 | pt. 1) | pt. 1) | pt. 1) | pt. 1) | of pt. 1) | pt. 1) |
| Age at time | 8 yo | 21 mo | 16 yo | 8 mo | 4 yo | 3 yo | 30 yo | 21 yo | 52 yo | 23 yo |
| of diagnosis | | | | | | | | | | |
| Sex | Male | Female | Male | Male | Male | Female | Female | Female | Female | Female |
| Ethnicity | Caucasian | Caucasian | Afro- | Hispanic | Caucasian | Caucasian | Caucasian | Caucasian | Caucasian | Caucasian |
| | | | American | | | | | | | |
| Cognitive | Severe | Global | Mild | Global | Global | Global | MR | MR | MR | Normal |
| function | MR, | DD | MR, | DD | DD | DD | | | | |
| | IQ = 21 | | IQ = 57 | | | | | | | |
| Seizures/ | None/ | None | None | None | None | None | Absence | Epilepsy | None | Epilepsy |
| EEG | abnormal | | | | | | seizures | since | | since age |
| | EEG | | | | | | | childhood | | 5 years |
| Inheritance | Maternal | Maternal | Unknown | Unknown | Maternal | Maternal | Maternal | Maternal | Unknown | Unknown |
| of CHRNA7 | | | (not | | | | | | | |
| deletion | | | paternal) | | | | | | | |
| Other CMA | None | dup | None | None | Unknown | Unknown | None | Unknown | Unknown | Unknown |
| abnormalities | | Xq26.2* | | | | | | | | |

CMA, chromosomal microarray analysis; DD, developmental delay; dup, duplication;

EEG, electroencephalography; IQ, intelligence quotient; mo, months, MR, mental retardation; pt., patient; yo, years.

*The ˜0.3 Mb duplication in Xq26.2 (chrX:131,336,391-131,600,358) is also maternally inherited.

The CHRNA7 deletion was inherited in two of two families where both parents were available for study. Propositus #1 is an 8-year-old Caucasian male with significant obesity (BMI=30.6) and severe mental retardation (Kaufman Assessment Battery for Children score of 21). Mild facial dysmorphisms in this patient include bilateral epicanthal folds, anteverted nares, and a thin upper lip. This individual has no history of seizures, but had an abnormal EEG with right temporo-occipital delta-wave deceleration and rhythmic 3-4 second delta activity. The deletion is present in his mother, two siblings, maternal aunt, and maternal grandmother (FIG. 4E). The mother and her sister have a history of mental retardation and epilepsy and the siblings and maternal grandmother have global developmental delay and mental retardation, respectively (FIG. 4E). Propositus #2 is a 21-month-old female with impaired growth (all growth parameters are below the 3rd percentile) and severe global developmental delay (no words and not walking at 21 months of age). Her CHRNA7 deletion is maternally inherited. The mother is reported to have normal intelligence, but with a history of epilepsy since the age of 5 years. Clinical information is sparse on both propositi #3 and #4. Patient #3 is a 16-year old Afro-American male with mild mental retardation (Intelligence quotient of 57), attention deficit hyperactivity disorder, and aggressive behavior. He has no history of seizures and EEG is reported as normal. His growth parameters are between the 50th-75^{th} percentiles. The patient's father does not carry the deletion, but the biological mother is unavailable for additional studies. Patient #4 was referred for genetic evaluation at the age of 8 months with global developmental delay, hypotonia, and failure to thrive. The inheritance of the CHRNA7 microdeletion is unknown for these two patients. In summary, four out of ten subjects with the CHRNA7 deletion manifest seizures or EEG abnormalities and nine out of ten present with developmental delay/mental retardation. The variable expressivity in our patients may relate to common polymorphisms on the remaining hemizygous allele, different genotypes elsewhere in the genome, and sex- and age-dependent penetrance for specific traits. Photographs of subjects were not provided, because of a report that deletion 15q13.3 is associated with antisocial behaviors (Ben-Schachar et al., 2009), which could stigmatize subjects and families.

The CHRNA7 deletion was found in the Medical Genetics Laboratories (MGL) at Baylor College of Medicine (BCM) in three of 8882 patients (1 in 2960) which is far rarer than the frequency of 1 in 350 of deletion 15q13.3 among mentally retarded individuals in the first published report1 and 1 in ˜530 samples referred to the MGL at BCM. In a series of 3,699 controls screened for copy number for CHRNA7 using SNP arrays and MLPA, no deletions were reported (Helbig et al., 2009). Remarkably, the inventor identified the reciprocal CHRNA7 duplication in 59 unrelated patients in 8882 subjects for a frequency of 1 in ˜170, close to a frequency of 1/185 among controls (Helbig et al., 2009). In all 21 of the CHRNA7 duplication cases where both parents were studied in our series, the duplication was inherited. Interestingly, in the same cohort, there were only three 1.5 Mb BP4-BP5 duplications. The high frequency of CHRNA7 duplications compared to deletions (59 vs. 3) and lack of de novo cases suggest that the duplication is either benign and/or associated with relatively high reproductive fitness. The proportion of inherited cases for both the larger deletion 15q13.3 (>60%) and the smaller deletion CHRNA7 is in sharp contrast to other deletions with a similar spectrum of phenotypes. For example, deletion 16p11.2 is a common cause of developmental delay, mental retardation and autism, but the vast majority of cases are of de novo origin (Weiss et al., 2008). This suggests that the reproductive fitness is relatively high for individuals with the 15q13.3 and CHRNA7 deletions.

In depth analysis of this genomic region revealed very complex arrangements of LCRs (Makoff and Flomen, 2007). However, all CHRNA7 deletions and duplications examined to date appear to have a near identical structure with a reciprocal pattern. The distal breakpoint maps within BP5, the proximal breakpoint between exons 1 and 2 of OTUD7A at the distal CHRNA7-LCR (FIG. 4A). Interestingly, the CHRNA7 deletion is always accompanied by an ˜90 kb duplication within BP4 (FIG. 4B,4C) while the reciprocal duplication is always associated with a deletion of the same region (FIG. 4D). In certain embodiments, the reciprocity of these rearrangements is due to NAHR between the LCR15q13.3 copies on a normal and inverted chromosome 15q13.3 in a cell heterozygous for the inversion (FIG. 4F); the inversion was shown to be present in high frequency in population (Sharp et al., 2008). To further characterize this, the inventor designed LCR-specific long-range PCR primers that would not amplify the original LCR15q13.3 copies but would amplify the predicted junction between these copies. In patients #2 and #3, the inventor amplified and sequenced the patient-specific PCR products that allowed one to narrow the NAHR sites between two informative cis-morphic nucleotides within 49 bp (chr15:28,815,284-28,815,332; chr15:29,749,424-29,749,472) and adjacent 67 bp (chr15:28,815,334-28,815,400; chr15:29,749,356-29,749,422) in the proximal and distal CHRNA7-LCRs, respectively (FIG. 1F). In patient #1, we narrowed the NAHR site to within 217 bp (chr15:28,776,996-28,777,212; chr15:29, 787,470-29,787,686) in the proximal and distal CHRNA7-LCRs, respectively. In certain embodiments, the BP4-BP5 inversion favors the meiotic NAHR between the CHRNA7-LCRs, perhaps explaining the high frequency of the CHRNA7 duplications. While there has been a rapid pace of discovery of disease-causing CNVs of larger size, many smaller pathological CNVs below 1 Mb likely remain to be delineated. Given (i) the known function of CHRNA7, (ii) the phenotypic overlap among patients with deletion 15q13.3 syndrome and CHRNA7 deletion, (iii) cosegregation in a family with six affected individuals, and (iv) the fact that this deletion was not seen in 3699 control samples (Helbig et al., 2009), haploinsufficiency of CHRNA7 is disease-causing. In support of this, mice lacking the α7 subunit of the neural nicotinic receptor exhibit impairment in working/episodic memory and in attentional processing (Hoyle et al., 2006).

The CHRNA7 protein is a target for rational drug design (Sharma and Vijayaraghavan, 2008) and is likely to be a very susceptible to alteration of function using agonists and antagonists. A naturally occurring agent, α-bungarotoxin, is a selective antagonist of the α7 nicotinic acetylcholine receptor in the brain and has long been used to study the nicotinic CNS system. A “proof-of-concept” trial has been performed in patients with schizophrenia using 3-[(2,4-dimethoxy)benzylidene]anabaseine (DMXB-A), a natural alkaloid derivative and a partial α7 nicotinic cholinergic agonist (Olincy et al., 2006).

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

### Example 3

### Diagnosis of Adverse Behavioral Condition

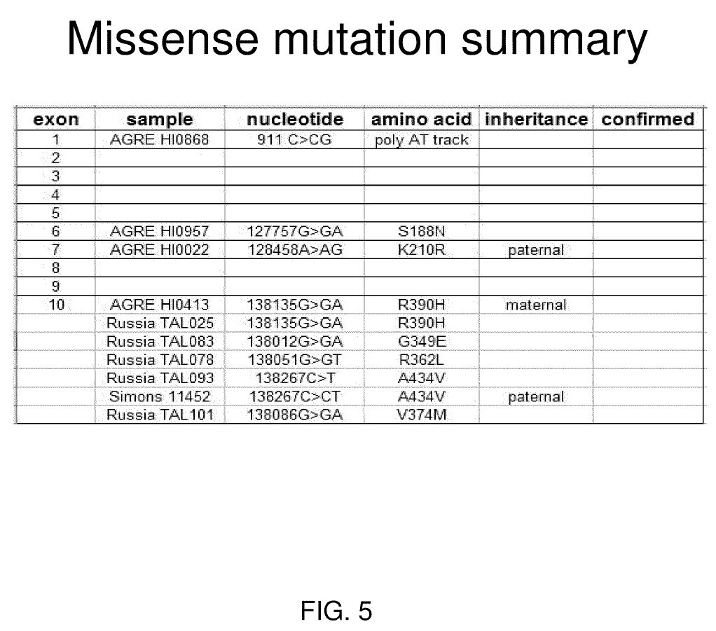

In certain embodiments of the invention, an individual exhibiting one or more symptoms of an adverse behavioral condition (that is at least not schizophrenia or any psychotic behavior, in certain cases) or at risk for having an adverse behavioral condition is subjected to testing for a mutation in the 15q13 locus. In specific cases, the individual is subjected to testing for a mutation in the CHRNA7 gene, including its coding region and/or regulatory region(s). In specific cases, the behavioral condition is violent behavior toward at least one other individual; psychopathy; or the individual is accused of being a sex offender or has been proven to be a sex offender or has been convicted of being a sex offender. In some cases, the mutation is a delection, such as a microdeletion. In specific embodiments, the mutation is a point mutation, missense mutation, frameshift mutation, or inversion. FIG. 5 illustrates individuals on death row from Russia that have missense mutations in CHRNA7 (see Russia TAL samples), whereas the other samples are from autism patients. One of skill in the art recognizes how to distinguish mutations between individuals based on the corresponding behavior exhibited by the individual.

In particular aspects, a sample from the individual is taken by the laboratory performing the assay, whereas in other cases the sample is obtained separately from the laboratory performing the assay. The sample may be hair, check scrapings, blood, skin, vaginal fluid, semen or so forth. In some cases, the sample is taken from the body of a victim of the individual.

In certain aspects, the information obtained by an assay for a mutation in 15q13 in an individual having one or more symptoms of an adverse behavioral condition is utilized in a court proceeding against the individual. The court proceeding may be a criminal or civil case, and the individual could be accused of breaking state and/or federal law.

### REFERENCES

Ballif, B. C., Hornor, S. A., Jenkins, E., Madan-Khetarpal, S., Surti, U., Jackson, K. E., Asamoah, A., Brock, P. L., Gowans, G. C., Conway, R. L. et al (2007). Discovery of a previously unrecognized microdeletion syndrome of 16p11.2-p12.2. Nat Genet 39, 1071-1073.
Ben-Shachar, S., et al. J. Med. Genet 46, 382-388 (2009).
Ben-Shachar, S., Ou, Z., Shaw, C. A., Belmont, J. W., Patel, M. S., Hummel, M., Amato, S., Tartaglia, N., Berg, J., Sutton, V. R. et al (2008). 22q11.2 distal deletion: a recurrent genomic disorder distinct from DiGeorge syndrome and velocardiofacial syndrome. Am J Hum Genet 82, 214-221.
Berg, J. S., Brunetti-Pierri, N., Peters, S. U., Kang, S. H., Fong, C. T., Salamone, J., Freedenberg, D., Hannig, V. L., Prock, L. A., Miller, D. T. et al (2007). Speech delay and autism spectrum behaviors are frequently associated with duplication of the 7q11.23 Williams-Beuren syndrome region. Genet Med 9, 427-441.
Biederer, T., and Sudhof, T. C. (2000). Mints as adaptors. Direct binding to neurexins and recruitment of munc18. J Biol Chem 275, 39803-39806.
del Gaudio, D., Fang, P., Scaglia, F., Ward, P. A., Craigen, W. J., Glaze, D. G., Neul, J. L., Patel, A., Lee, J. A., Irons, M. et al (2006). Increased MECP2 gene copy number as the result of genomic duplication in neurodevelopmentally delayed males. Genet Med 8, 784-792.
Elmslie, F. V., Rees, M., Williamson, M. P., Kerr, M., Kjeldsen, M. J., Pang, K. A., Sundqvist, A., Friis, M. L., Chadwick, D., Richens, A. et al (1997). Genetic mapping of a major susceptibility locus for juvenile myoclonic epilepsy on chromosome 15q. Hum Mol Genet 6, 1329-1334.
Fernandes, C., Hoyle, E., Dempster, E., Schalkwyk, L. C., and Collier, D. A. (2006). Performance deficit of alpha7 nicotinic receptor knockout mice in a delayed matching-to-place task suggests a mild impairment of working/episodic-like memory. Genes Brain Behav 5, 433-440.
Flomen, R. H., Collier, D. A., Osborne, S., Munro, J., Breen, G., St, C. D., and Makoff, A. J. (2006). Association study of CHRFAM7A copy number and 2 bp deletion polymorphisms with schizophrenia and bipolar affective disorder. Am J Med Genet B Neuropsychiatr Genet 141B, 571-575.
Flomen, R. H., Davies, A. F., Di, F. M., Cascia, C. L., kie-Ogilvie, C., Murray, R., and Makoff, A. J. (2008). The copy number variant involving part of the alpha7 nicotinic receptor gene contains a polymorphic inversion. Eur J Hum Genet [Epub ahead of print]
Freedman, R., Coon, H., Myles-Worsley, M., Orr-Urtreger, A., Olincy, A., Davis, A., Polymeropoulos, M., Holik, J., Hopkins, J., Hoff, M. et al (1997). Linkage of a neurophysiological deficit in schizophrenia to a chromosome 15 locus. Proc Natl Acad Sci USA 94, 587-592.
Freedman, R., Leonard, S., Gault, J. M., Hopkins, J., Cloninger, C. R., Kaufmann, C. A., Tsuang, M. T., Farone, S. V., Malaspina, D., Svrakic, D. M. et al (2001). Linkage disequilibrium for schizophrenia at the chromosome 15q13-14 locus of the alpha7-nicotinic acetylcholine receptor subunit gene (CHRNA7). Am J Med Genet 105, 20-22.
Gault, J., Robinson, M., Berger, R., Drebing, C., Logel, J., Hopkins, J., Moore, T., Jacobs, S., Meriwether, J., Choi, M. J. et al (1998). Genomic organization and partial duplication of the human alpha7 neuronal nicotinic acetylcholine receptor gene (CHRNA7). Genomics 52, 173-185.
Gordon, A. R., Outram, S. V., Keramatipour, M., Goddard, C. A., Colledge, W. H., Metcalfe, J. C., Hager-Theodorides, A. L., Crompton, T., and Kemp, P. R. (2008). Splenomegaly and modified erythropoiesis in KLF13−/− mice. J Biol Chem 283, 11897-11904.
Helbig, I., et al. Nat. Genet. 41, 160-162 (2009).
Ho, A., Morishita, W., Atasoy, D., Liu, X., Tabuchi, K., Hammer, R. E., Malenka, R. C., and Sudhof, T. C. (2006). Genetic analysis of Mint/X11 proteins: essential presynaptic functions of a neuronal adaptor protein family. J Neurosci 26, 13089-13101.
Hoyle, E., et al. Psychopharmacology (Berl.) 189, 211-223 (2006).
Iafrate, A. J., Feuk, L., Rivera, M. N., Listewnik, M. L., Donahoe, P. K., Qi, Y., Scherer, S. W., and Lee, C. (2004). Detection of large-scale variation in the human genome. Nat Genet 36, 949-951.
International Schizophrenia Consortium Nature 455, 237-241 (2008).
Jacquemont, M.-L., Sanlaville, D., Redon, R., Raoul, O., Cormier-Daire, V., Lyonnet, S., Amiel, J., Le, M. M., Heron, D., De Blois, M.-C. et al (2006). Array-based comparative genomic hybridization identifies high frequency of cryptic chromosomal rearrangements in patients with syndromic autism spectrum disorders. J Med Genet 43, 843-849.
Kayagaki, N., Phung, Q., Chan, S., Chaudhari, R., Quan, C., O'Rourke, K. M., Eby, M., Pietras, E., Cheng, G., Bazan, J. F. et al (2007). DUBA: a deubiquitinase that regulates type I interferon production. Science 318, 1628-1632.
Kim, H. G., Kishikawa, S., Higgins, A. W., Seong, I. S., Donovan, D. J., Shen, Y., Lally, E., Weiss, L. A., Najm, J., Kutsche, K. et al (2008). Disruption of neurexin 1 associated with autism spectrum disorder. Am J Hum Genet 82, 199-207.
Kirov, G., Gumus, D., Chen, W., Norton, N., Georgieva, L., Sari, M., O'Donovan, M. C., Erdogan, F., Owen, M. J., Ropers, H. H. et al (2008). Comparative genome hybridization suggests a role for NRXN1 and APBA2 in schizophrenia. Hum Mol Genet 17, 458-465.
Kunert-Keil, C., Bisping, F., Kruger, J., and Brinkmeier, H. (2006). Tissue-specific expression of TRP channel genes in the mouse and its variation in three different mouse strains. BMC Genomics 7, 159.
Leonard, S., & Freedman, R. Biol. Psychiatry 60, 115-122 (2006).
Lounsbury, D. W., Reynolds, T. C., Rapkin, B. D., Robson, M. E., and Ostroff, J. (2007). Protecting the privacy of third-party information: recommendations for social and behavioral health researchers. Soc Sci Med 64, 213-222.
Lu, X., Shaw, C. A., Patel, A., Li, J., Cooper, M. L., Wells, W. R., Sullivan C. M., Sahoo, T., Yatsenko, S. A., Bacino, C. A. et al (2007). Clinical implementation of chromosomal microarray analysis: summary of 2513 postnatal cases. PLoS ONE 2, e327.
Lupski, J. R. (1998). Genomic disorders: Structural features of the genome can lead to DNA rearrangements and human disease traits. Trends Genet 14, 145-149.
Lupski, J. R., (2007). Genomic rearrangements and sporadic disease. Nat Genet 39, S43-S47.
Makoff, A. J., and Flomen, R. H. (2007). Detailed analysis of 15q11-q14 sequence corrects errors and gaps in the public access sequence to fully reveal large segmental duplications at breakpoints for Prader-Willi, Angelman, and inv dup(15) syndromes. Genome Biol 8, R114.
Marshall, C. R., Noor, A., Vincent, J. B., Lionel, A. C., Feuk, L., Skaug, J., Shago, M., Moessner, R., Pinto, D., Ren, Y. et al (2008). Structural variation of chromosomes in autism spectrum disorder. Am J Hum Genet 82, 477-488.
Martin, L. F., and Freedman, R. (2007). Schizophrenia and the alpha7 Nicotinic Acetylcholine Receptor. Int Rev Neurobiol 78, 225-246.
Miller, D. T., et al. J. Med. Genet. 46, 242-248 (2009).
Morrow, E. M., Yoo, S.-Y., Flavell, S. W., Kim, T.-K., Lin, Y., Hill, R. S., Mukaddes, N. M., Balkhy, S., Gascom, G., Hashmi, A. et al (2008). Identifying autism loci and genes by tracing recent shared ancestry. Science 321, 218-223.
Murphy, K. C., (2002). Schizophrenia and velo-cardio-facial syndrome. Lancet 359, 426-430.
Neubauer, B. A., Fiedler, B., Himmelein, B., Kampfer, F., Lassker, U., Schwabe, G., Spanier, I., Tams, D., Bretscher, C., Moldenhauer, K. et al (1998). Centrotemporal spikes in families with rolandic epilepsy: linkage to chromosome 15q14. Neurology 51, 1608-1612.
Olincy, A., et al. Arch. Gen. Psychiatry 63, 630-638 (2006).
Orr-Urtreger, A., Göldner, F. M., Saeki, M., Lorenzo, I., Goldberg, L., De Biagi, M., Dani, J. A., Patrick, J., and Beaudet, A. L. (1997). Mice deficient in the a7 neuronal nicotinic acetylcholine receptor lack a-bungarotoxin binding sites and hippocampal fast nicotinic currents. J Neurosci 17, 9165-9171.
Ou, Z., Kang, S. H., Shaw, C. A., Carmack, C. E., White, L. D., Patel, A., Beaudet, A. L., Cheung, S. W., and Chinault, A. C. (2008). Bacterial artificial chromosome-emulation oligonucleotide arrays for targeted clinical array-comparative genomic hybridization analyses. Genet Med 10, 278-289.
Outram, S. V., Gordon, A. R., Hager-Theodorides, A. L., Metcalfe, J., Crompton, T., and Kemp, P. (2008). KLF13 influences multiple stages of both B and T cell development. Cell Cycle 7, 2047-2055.
Potocki, L., Bi, W., Treadwell-Deering, D., Carvalho, C. M., Eifert, A., Friedman, E. M., Glaze, D., Krull, K., Lee, J. A., Lewis, R. A. et al (2007). Characterization of Potocki-Lupski syndrome (dup(17)(p11.2p11.2)) and delineation of a dosage-sensitive critical interval that can convey an autism phenotype. Am J Hum Genet 80, 633-649.
Ray, M. A., Graham, A. J., Lee, M., Perry, R. H., Court J A, and Perry, E. K. (2005). Neuronal nicotinic acetylcholine receptor subunits in autism: an immunohistochemical investigation in the thalamus. Neurobiol Dis 19, 366-377.
Resnik, D. B., and Sharp, R. R. (2006). Protecting third parties in human subjects research. IRB 28, 1-7.
Sahoo, T., Peters, S. U., Madduri, N., Glaze, D. G., German, J. R., Bird, L., Barbierei-Welge, R., Bichell, T. J., Beaudet, A. L., and Bacino, C. A. (2006). Microarray-based comparative genomic hybridization testing in deletion-bearing Angelman syndrome patients: genotype-phenotype correlations. J Med Genet 43, 512-516.
Sahoo, T., Bacino, C. A., German, J. R., Shaw, C. A., Bird, L. M., Kimonis, V., Anselm, I., Waisbren, S., Beaudet, A. L., and Peters, S. U. (2007). Identification of novel deletions of 15q11q13 in Angelman syndrome by array-CGH: molecular characterization and genotype-phenotype correlations. Eur J Hum Genet 15, 943-949.
Sahoo, T., del Gaudio, D., German, J. R., Shinawi, M., Peters, S. U., Person, R. E., Garnica, A., Cheung, S. W., and Beaudet, A. L. (2008). Prader-Willi phenotype caused by paternal deficiency for the HBII-85 C/D box small nucleolar RNA cluster. Nat Genet 40, 719-721.
Sebat, J., Lakshmi, B., Troge, J., Alexander, J., Young, J., Lundin, P., Månér, S., Massa, H., Walker, M., Chi, M. et al (2004). Large-scale copy number polymorphism in the human genome. Science 305, 525-528.
Sebat, J., Lakshmi, B., Malhotra, D., Troge, J., Lese-Martin, C., Walsh, T., Yamrom, B., Yamrom, B., Yoon, S., Krasnitz, A. et al (2007). Strong Association of De Novo Copy Number Mutations with Autism. Science 316, 445-449.
Sharma, G., & Vijayaraghavan, S. Curr. Med. Chem. 15, 2921-2932 (2008).
Sharp, A. J., Hansen, S., Selzer, R. R., Cheng, Z., Regan, R., Hurst, J. A., Stewart, H., Price, S. M., Blair, E., Hennekam, R. C. et al (2006). Discovery of previously unidentified genomic disorders from the duplication architecture of the human genome. Nat Genet 38, 1038-1042.
Sharp, A. J., Locke, D. P., McGrath, S. D., Cheng, Z., Bailey, J. A., Vallente, R. U., Pertz, L. M., Clark, R. A., Schwartz, S., Segraves, R. et al (2005). Segmental duplications and copy-number variation in the human genome. Am J Hum Genet 77, 78-88.
Sharp, A. J., Mefford, H. C., Li, K., Baker, C., Skinner, C., Stevenson, R. E., Schroer, R. J., Novara, F., De, G. M., Ciccone, R. et al (2008). A recurrent 15q13.3 microdeletion syndrome associated with mental retardation and seizures. Nat Genet 40, 322-328.
Stankiewicz, P., and Beaudet, A. L. (2007). Use of array CGH in the evaluation of dysmorphology, malformations, developmental delay, and idiopathic mental retardation. Curr Opin Genet Dev 17, 182-192.
Stankiewicz, P., and Lupski, J. R. (2002). Genome architecture, rearrangements and genomic disorders. Trends Genet 18, 74-82.
Stefansson, H., Rujescu, D., Cichon, S., Pietilainen, O. P., Ingason, A., Steinberg, S., Fossdal, R., Sigurdsson, E., Sigmundsson, T., Buizer-Voskamp, J. E. et al, Nature 455, 232-236 (2008). Large recurrent microdeletions associated with schizophrenia. Nature [Epub ahead of print]
Stephens, S. H., et al. Schizophr. Res. 109, 102-112 (2009).
Stone, J. L., O'Donovan, M. C., Gurling, H., Kirov, G. K., Blackwood, D. H., Corvin, A., Craddock, N. J., Gill, M., Hultman, C. M., Lichtenstein, P. et al (2008). Rare chromosomal deletions and duplications increase risk of schizophrenia. Nature [Epub ahead of print]
Taske, N. L., et al. Epilepsy Res. 49, 157-172 (2002).
Tronchere, H., Buj-Bello, A., Mandel, J. L., and Payrastre, B. (2003). Implication of phosphoinositide phosphatases in genetic diseases: the case of myotubularin. Cell Mol Life Sci 60, 2084-2099.
Van, E. H., Bauters, M., Ignatius, J., Jansen, M., Raynaud, M., Hollanders, K., Lugtenberg, D., Bienvenu, T., Jensen, L. R., Gecz, J. et al (2005). Duplication of the MECP2 region is a frequent cause of severe mental retardation and progressive neurological symptoms in males. Am J Hum Genet 77, 442-453.
van Bon, B. W., et al. J. Med. Genet. [Epub ahead of print], (2009).
Vorstman, J. A. S., Staal, W. G., van, D. E., van, E. H., Hochstenbach, P. F. R., and Franke, L. (2006). Identification of novel autism candidate regions through analysis of reported cytogenetic abnormalities associated with autism. Mol Psychiatry 11, 1, 18-1, 28.
Walsh, T., McClellan, J. M., McCarthy, S. E., Addington, A. M., Pierce, S. B., Cooper, G. M., Nord, A. S., Kusenda, M., Malhotra, D., Bhandari, A. et al (2008). Rare structural variants disrupt multiple genes in neurodevelopmental pathways in schizophrenia. Science 320, 539-543.
Wang, N. J., Liu, D., Parokonny, A. S., and Schanen, N. C. (2004). High-resolution molecular characterization of 15q11-q13 rearrangements by array comparative genomic hybridization (array CGH) with detection of gene dosage. Am J Hum Genet 75, 267-281.
Weiss, L. A., Shen, Y., Korn, J. M., Arking, D. E., Miller, D. T., Fossdal, R., Saemundsen, E., Stefansson, H., Ferreira, M. A., Green, T. et al (2008). Association between microdeletion and microduplication at 16p11.2 and autism. N Engl J Med 358, 667-675.
Xu, B., Roos, J. L., Levy, S., van Rensburg, E. J., Gogos, J. A., and Karayiorgou, M. (2008). Strong association of de novo copy number mutations with sporadic schizophrenia. Nat Genet 40, 880-885.

What is claimed is:

1. A method of identifying an individual that has or is predisposed to having one or more adverse behavioral conditions, comprising the step of analyzing nucleic acid from chromosome 15q13 locus from the individual, wherein the individual is or exhibits behavior associated with one or more of the following: violent criminal behavior, sex offender, psychopathy, aggressive behavior, antisocial behavior, antisocial personality disorder, borderline personality disorder, conduct disorder, intermittent explosive disorder, paranoid personality disorder, paraphilia, posttraumatic stress disorder, pyromania, schizoid personality disorder, schizotypal personality disorder, substance abuse, and substance dependence, wherein when the individual has a mutation in the locus, the individual has or is predisposed to having the adverse behavioral condition.

2. The method of claim 1, further defined as analyzing one or more of CHRNA7, OTUD7A, KLF13, TRPM1, MTMR10, MTMR15, CHRFAM7A or FAM7A(2).

3. The method of claim 1, wherein the individual is a sex offender or exhibits violent criminal behavior or psychopathy.

4. The method of claim 1, wherein the mutation is a microdeletion.

5. The method of claim 1, further comprising the step of utilizing the information from the analyzing step in a criminal proceeding under federal or state law.

6. The method of claim 1, wherein the individual is a child or an adult.

7. The method of claim 1, wherein the individual has already exhibited one or more of the following behaviors: aggression toward another, disregard for well-being of self or others, lack of remorse, deviant sexual behavior, uninhibited gratification in criminal, sexual, or aggressive impulses, or the inability to learn from past mistakes.

8. The method of claim 1, wherein the individual has been convicted of one or more of homicide, rape, assault, arson, or battery.

9. The method of claim 1, wherein the analyzing utilizes long-range polymerase chain reaction.

10. A method of identifying an individual that is predisposed to criminal or antisocial behavior, comprising the step of analyzing chromosome 15q13 locus from said individual.

11. The method of claim 10, wherein said criminal or antisocial behavior comprises violence, aggression, sex offense, and/or drug abuse.

12. The method of claim 10, wherein the individual is an infant or child.

13. The method of claim 10, further defined as assaying for a microdeletion in the 15q13 locus.

14. The method of claim 10, further defined as analyzing one or more of CHRNA7, OTUD7A, KLF13, TRPM1, MTMR10, MTMR15, CHRFAM7A or FAM7A(2).

15. The method of claim 14, wherein CHRNA7 is analyzed.

16. The method of claim 14, wherein a microdeletion in CHRNA7 is analyzed.

17. The method of claim 10, further comprising the step of utilizing the information from the analyzing step in a criminal proceeding under federal or state law.


SEQUENCE LISTING (8 sequences)

SEQ ID NO: 1 - DNA, 2171 bp, Human
  CHRNA7
ggccgcaggcgcaggcccgggcgacagccgagacgtggagcgcgccggctcgctgcagctccgggactcaacatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacagtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagcgccgtggcgccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctttggcttggcgagatttgggggtgctaatccaggacagcattacacgccacaactccagtgttcccttctggctgtcagtcgtgttgcttacggtttctttgttactttaggtagtagaatctcagcactttgtttcatattctcagatgggctgatagatatccttggcacatccgtaccatcggtcagcagggccactgagtagtcattttgcccattagcccactgcctggaaagcccttcggagagctccccatggctcctcaccaccgagacagttggttttgcatgtctgcatgaaggtctacctgaaaattcaacatttgctttttgcttgtgtacaaacccagattgaagctaaaataaaccagactcactaaatcctttccaataattgactggtggaaggaaaacaaaaaacaaaaactaaaaacctcttagcttttctgcaattcaactttttatttttatttttatttctatcaaagacggtagagagaaacagcttgatgctgtttctacattaaaaaaaaaaaaaaaa

SEQ ID NO: 2 - DNA, 3042 bp, Human
  OTUD7A
gttctctcgctcaggtctctgcatgtagttgtcacttgcagctccatttccatcacgtggtaaaatgccctttctcttctttcctgcagatggatggtttctagtgtgcttccaaaccccacctcggctgagtgttgggcagcacttctacatgatcctatgactcttgatatggacgcagtcctgtcagactttgttcggtccacgggggcagaacctggtctggccagagacctgctggaaggcaaaaactgggacctgacagccgctctgagcgactatgagcagctccgccaggtgcacacagccaatctgccacatgtgttcaatgaagggcggggtcccaagcagccagagcgagagccacagcccgggcacaaggtggagcgaccctgcctgcagaggcaggacgacattgcccaagaaaagcggctttcccgggggatttcccacgccagctcagccatcgtctccctggcccggtcccacgtggcaagtgaatgcaacaacgagcagttccccctggagatgccaatctacacattccagttgccagacctgagcgtgtacagcgaggatttcaggagcttcatcgagcgggacttgatcgagcaggcaacaatggtggctttggagcaggcaggtcgcctgaactggtggtccactgtgtgcacgagctgtaaacggcttcttcctctggccacaacaggggatgggaactgccttttacatgctgcttcactgggaatgtgggggtttcacgaccgggacctggtgttacggaaagctctctataccatgatgaggacgggagctgagagggaagccctgaagcggaggtggaggtggcagcagacgcagcagaataaggaggaggagtgggagcgggagtggacggagctgctgaagctggcctccagcgagccgcgcacacacttcagcaagaatggcggcacgggcgggggtgtggacaactctgaggaccccgtgtacgagagcctggaagagttccacgtttttgtcctagcccatatattaagaaggcccatcgttgttgtggcagatacaatgttaagagactcaggtggagaagcgttcgcacccatcccattcggagggatctacctgcccttggaggtccctcccaacagatgccactgctcgcctctggttctggcctatgatcaagcccatttctctgcccttgtgtccatggaacagagagaccagcaaagagaacaagccgtgatccccctgacggattctgagcacaagctgctgcctctgcactttgcagtggaccctggcaaggactgggagtgggggaaagacgacaacgataacgcccggctggcccaccttatcctgtcgctagaagccaagctgaaccttctgcacagctacatgaacgtgacgtggatccggatcccctccgagacacgggcgcccctggcacagccggagtctcccacggcctcggcaggggaggacgtgcagtccctggccgactcgctggactcggaccgcgattcggtgtgcagcaattctaacagcaataacggcaagaacggcaaggacaaggagaaggagaagcagcgcaaggagaaggacaagacgcgcgccgactccgtggccaacaagctgggcagcttcagcaagacgctgggcatcaagctgaagaaaaacatgggcggcctcggcggcctggtgcacggcaagatgggccgcgccaactccgccaatggcaagaacggggactcggccgagcggggcaaggagaagaaggccaagtcgcgcaagggcagcaaggaggagtctggtgcgtcggccagcacgtcgccgtcggaaaagaccacgccgtcgcccacagacaaggcagcgggcgcgtcgccggcggagaagggcggtgggccgcggggcgacgcctggaagtacagcacggatgtgaagctgagcctcaacatcctgcgcgccgccatgcagggggagcgcaagttcatcttcgccggcctgctgctcaccagccaccggcaccagttccacgaggagatgatcggctactacctgacgagcgcgcaggagcgcttcagcgccgagcaggagcagcggcgccgcgacgccgctactgcggccgccgctgccgccgccgccgccgccgccacggccaagcggccgccgcgcagaccggagacggagggcgtgccggtcccggagcgcgcctctccgggcccacccacgcagctggtgctcaagctcaaggagcggccgagccccgggcccgcggcagggcgtgcggcgcgggcggcggcgggcggcacggcctccccggggggaggcgcgcggcgtgcgagcgccagcggaccagtgcctggccgcagccccccggcgccagcgcgccagagcgtcatccacgtgcaggcgtcgggcgcgcgggacgaggcgtgcgcgccggccgtgggggcgctgcggccgtgcgccacgtacccgcagcagaaccgctcgctgtcgtcgcagagctacagcccggcgcgcgccgccgccctgcgcaccgtcaacacggtcgagtcgctggcgcgcgcggtgcccggggccctaccgggcgcggcggggacggcgggggcggccgagcacaagtcgcagacctacaccaacggcttcggcgccctgcgcgacggcctggagttcgccgacgccgacgcgccgaccgcgcgctcgaacggtgagtgcggccgtggcggcccggggccggtgcagcggcgctgccagcgcgagaactgtgcgttctacgggcgcgccgagaccgagcactactgctcctactgctaccgcgaggagctgcggcggcggcgcgaggcgcgcggggcccggccctgagcggcgcggcgcggcgcgggcggcgaggttctaccttcgaggatttcttttccattgtgtcggtgtcttttttacatgccctggtccaccggaaggccggcgcctcctctgtcagtgccgtgtacgtgtttggtcaaacgttcctaatggtgcctgaacctacactga

SEQ ID NO: 3 - DNA, 6817 bp, Human
  KLF13
gcgcggcgaagctgcggccgcgacttggcgaggtggtccctaacgttgccgctcggcatccttagaaccggccgcccctgacgccgcgcggggaccccagtcgcccgcgcgccccatgcgctcactcttcggtgcccggccgggccggcgcctcgcagacgcggagccgcgcgggtgacggcacaggcggctgcgcgcccagcccagcccagcccagcccgaggagagggcgcgccgcgcccccgccccccgcccgctctcccgaggccgtgggtgcggatgcgcggctgacgactcgcagcaagagcaccgccgccggccccagcccgcagcatggcagccgccgcctatgtggaccacttcgccgccgagtgcctcgtgtccatgtcgagccgcgcggtcgtgcacgggccgcgggaggggccggagtcccggcccgagggcgcggccgtggccgccacccccacgctgccccgcgtcgaggagcgccgcgacggtaaggacagcgcctcgctcttcgtggtggcgcggatcctagcggacctcaaccagcaagcgccggcgcccgccccggcggagcgcagggagggcgccgcggcccggaaggcgaggaccccctgccgcctgccgccgcccgcccccgagcccacctcccccggcgccgaaggcgcggcggccgcgccccccagcccggcgtggagcgagccggagcccgaggcggggctggagcccgagcgggagccggggcccgcggggagcggcgagcccggcctcagacaaagggtccggcggggccgaagtcgcgccgacctcgagtccccgcagaggaagcacaagtgccactacgcgggctgcgagaaagtttacgggaaatcttcgcacctcaaggcgcacctgagaactcacacaggtgagaggcccttcgcctgcagctggcaggactgcaacaagaagttcgcgcgctccgacgagctggcgcggcactaccgcacacacacgggcgagaagaagttcagctgccccatctgcgagaagcgcttcatgcgcagcgaccacctgaccaagcacgcgcgccgccacgccaacttccacccgggaatgctgcagcggcgcggcgggggctcgcggaccggctccctcagcgactacagccgctccgacgccagcagccccaccatcagcccggccagctcgccctgagcccgccacagccatgagcagccgctcccaccccctcgtgagtccctggcctttccttttgtaataagaaagaagagagagaacttgatgcaaagtccacgaaaaaacaatttttttcacctcaggtgtcaaagtaaatttgttaaaaaaacaaaaaaaacacaaaaatttcaaaaaacaccacccaccaaattgcacaatagatacacccacaaagttgagattcagcgttgttgaaccccctttctcagggatggacacgtttcacgaggtcagtgaggacaccccttcctgccgccttactctgtacatagatttgcactctggagttttctgttaggttcgggaacacgctggggacagagcaggccagccagtctggtggagctcagcgtctggctggctggccagcctgtgggtctgttgggagcagatgtgctcactgacgtttgtgcctccagggcagattcctaagcagctggcctgacctcttcctgcccagcccccaacccagtctctggcctctgcccaacgagcctggctgggcttgcaacgcagctgattctgagacaggcccctgcagagggatgccttaaagctgtccctggctggaggagcactccaccttggggatagtggagggggccttcagccctctgcaccctgcctgcctcacaccccgccttcaggagccctctcctacggcgctagggagcactggaagcagagatgccagccctggctgagcgtagcactgcaccgtccgacctccgaggcaggggccggggtcacacctgccaggactccgcttctccgtgtcccagcagaaggcatgcaggaccctgtcactactatacctgggcctctgatggggaattctggtcttctaaaaagatgttagaaattcctggtggcacatccagttccgagtttgcccgcatgggcacttttgttggaaacagtgggcaggtcatggggcctcctcatcagagggtctgtgtgagcggctgtgcatgtggcagcgcaccatgcttcacagagaccattctgtagcaagagaccggaacattgtgacttggaccttttcaggagtggcgcggatgttacaggaaatgctgtcggagagcgcgcattctatttcctccgcagggaatgccagatagaaagttgcgggcaacacttttctcagacccaccatgtccccaactcagacttagcaaacctccagcctctccgtttcccctggactcctcctcccttttcctcctgcttctccttgtcctgcctggaggctccaggccacacccagctggtggggctgctccagtgccccatcaccggccttctcctggcaggagaggaggagcagagagctgggtcctgggctgggattcgagccctgggcgggggtcagtcaagccgtggtccagcaaactcctctgcagatggctggagtgttgacccaggcttgtggcccaggtatcaccttcccttcctcctgagagttgaggcctgtaaatacaagggcccaggacagagtgtgtgcgtgcgtgtgggtgtgtgcgcgcgtgagcacacacgcgtgtgttggggggggtggggggattggggtggactcagggtatcttgccagagatacctgttttgatgagtacctattttgatgcaaaggaacggccctgggacctcagagggccacagatgggcttcctgggcctagcgtgccttggttgtggccatcctgcttctactcagccctgccatgctatggggtgcagcacaaacccaggacccaggcctagccactgcccacagctgatggtcactgttttggctggctccagagtggacaggtacgctctgtccacagcagctgctcaggcacagtatgccgtcaggtgcccgcttcttaccactgtgtgtcagctgggattggccgacagcgtggtggtgacttctaggtagaccgatggaccctaaattgggcagtggcattgtttctgctgtggtgggaactggcagggagagggtcacctccactctggttggatgcggagctggtccctctggctgccatgctgtgttggggctctctgacctccctgcttgcattgggtctgggagagagagtggaatatttggtagggaccttgacagaagagagccctgggtgcttgtggtgcaaagatcttcatgcccagttccaaagtgaggtccctcccaaagttgttttcgtgtgaggcagagaaagccacactctgaatacattttatgtctcaggaattcacattccaggttcaggaattttattccaaagtcctttggtgcactcaccgtccaccgttccccaaggcagggagagggagtggcccgtccctgggaggctggcaaagtgctctccagggaggggtcagacgtccttgtgatctgcccagggtgtcacggaaggagattttctagctccacatctgtctgcacataggaaggaaaagataggactctaaacctagtcatcttggaataaaaagaagagctggtttgttttagagcagggcggggggtgggggagggaagcgggtgttggctgtttccaaagagagcacttaatttaatttttcctttaaatgacccagggctgttccgtctgatagatggaagggtaacattgccttaaaacagaaatatgcaggcgttggctatttttggcataagagcgtgtcttcattcccaaagtggttctcgtttaatggcagggtgcggtccttaggccccggcctggactgggaaatgggggtagggcaccaggagagaaatcctcctgggtactcctggccagaccctcctgaatcatctgcggcttaaactcatctcgcttgaagggcagggagaaacccatgaatctcaggcacggttcacaggggtcctcaagggcaggtttccatttcatcatgaagtgagggactcctgtggaagagggtggtgggccttggaacgctgcctccactgaggcgggcacacaaggcggaccctcctgaaacagcctccattttcttcaaacatgcagaatgttttcccagactgcgtctatttcagggtcttccatttttgtggtttatggccatgcttgccagttttggtggggttgaggaacccataggactcatagaaataatagtgaaaactcaaacccttattttgggacttcagttctcgtggtccataggggcagagatgtcaaagtgaaggttgcctgagtgaagggagagttcccctataaaaggcctaaaattgatctggggctcagccatcaaggggctggtgttgctcctgtcccagacaaggaagcccctctttacatcagccatattaggggaacggttgtcagctccccaaatgcctactggatcctacagccatacaggaaaacagacggacacagccctttccttaaagagccctggggtcctgtcctgcaggatgctgcataggaatgggcagagatgtcctggatgggaacatggtcctggaggtgccctacctgcatcctgctgcacccccacccacgacactcgagtgcaagatcgcctcactcctaaccctgactttattcccaggccccacaccgaggcaggtgcaggattcgccatggtgcagcctccgggtacaaacatctgaggcactggcgatcccagtccttgcagcaccctgaacacagctcagaccagtgaggggtgattcagaggtggggatgggcagagagggctggccactctgcctcaggacttgacctcaagtgttctgtctccccacccggtgtgtccagaagaccagcctctttcttgccagaagcaggtgctaagatggactgcacagggtcccggcactccctcgggacccattcaggatctcagctgggcaggggtgccacccaacaggaagatgtgatctgagcaatagtgtggccttgtttctccgcttctggttgtggctgtgtgtacagggctacctattttgacttctgacaggtgtcactgattttctttcccccatttaaaaaaggtcatttagtcaaagacacagtgctagaggtttaaagggtgtaatgtgttggtcacatcacaaccagtaagaaacgcttggtaggttatccacatcttttgctgactgtttccatatacaattctggtcaccatattttccaaagctaaaatctgaggcgtgggctgatggtttttgaaatcaggattaccccaggaagaaccaggtcctattactaattttcctgattctgaggctctgaaggcttgtctgagacacattccggagacctttgtaccagaacttggatgcatagtccaccttactactgatacacgcctgagcaccctttagggcgaggcgccgcctcctgcctgctgtcctgtccctcagcatcagaagagttgaattcagcaggagacgggagcccggtgtccatgagggaagaggggtcgcgcttggtttctactgacccccatgaagatttcagacttgcagtgcggccacctggccctgccctgcctctctctttctcatacagctttaaaactttactacttttatttaaaaatgaactggatgggagagaagtagcgtcccctaccctacaagtcacacattccggggaggggggtggggggtggaggcaggaagtcatgggggtgggggtgggagcacgggaacagctttcttaaggcctcagggtcctgttttccctggcctcttctagagggcccgtggacaggtcgcagtgcgtgcttatttggaaaccaggtgtgtgagccgaatgcctgccaggccatgcactcagcagaggagcccttgtacctggctgccctgagaggagcaagaggccacctcccatgtggctctagacaccacgtgggctcattagccccagcgtctgtgccggctccaggtgcctgcctctgggtgtgtgagtgggagactttgctccctggcctcatcctagagaggcccctggtgcctagtgctgaggcctctggggctggaaagcctcagcagaaaggaggcactactgagcaactatgcattgtcattgtcgggtttggggctttcggtggttccttggtgactgggaattgcttgtgtgcatgtgttgggtgcatgcttccgggtctcagctgccccaggcccgcacaggcaaccccttcccatccaaagccattggtggagcttctctggaatcatttgccaaaagcccaaggcagaatccaagggtccaagaccatttccatggagctcatgtttttcttttctgtaggaacttttttttaaccagcacccaccataattccgaaggccacgtttcatctttcctggatcactacagtgaagtattacagttgtacagttcccagtctggccttggcttgctcggataaaactttgtatgtattttgtatggcatagattctatattgtaatgatgtcctatgcaaaaagaaaaattaacgaaattgtaaattttattgttttaacgtgtatgcatgtttagtgacgtttacattttgaaataaaatttatgattcattattttaaaaaaaaaaaaaaaaaa

SEQ ID NO: 4 - DNA, 5428 bp, Human
  TRPM1
gccctggccaaggaggaggctgaaagagcctgagctgtgccctctccattccactgctgtggcagggtcagaaatcttggatagagaaaaccttttgcaaacgggaatgtatctttgtaattcctagcatgaaagactctaacaggtgttgctgtggccagttcaccaaccagcatatcccccctctgccaagtgcaacacccagcaaaaatgaagaggaaagcaaacaggtggagactcagcctgagaaatggtctgttgccaagcacacccagagctacccaacagattcctatggagttcttgaattccagggtggcggatattccaataaagccatgtatatccgtgtatcctatgacaccaagccagactcactgctccatctcatggtgaaagattggcagctggaactccccaagctcttaatatctgtgcatggaggcctccagaactttgagatgcagcccaagctgaaacaagtctttgggaaaggcctgatcaaggctgctatgaccaccggggcctggatcttcaccgggggtgtcagcacaggtgttatcagccacgtaggggatgccttgaaagaccactcctccaagtccagaggccgggtttgtgctataggaattgctccatggggcatcgtggagaataaggaagacctggttggaaaggatgtaacaagagtgtaccagaccatgtccaaccctctaagtaagctctctgtgctcaacaactcccacacccacttcatcctggctgacaatggcaccctgggcaagtatggcgccgaggtgaagctgcgaaggctgctggaaaagcacatctccctgcagaagatcaacacaagactggggcagggcgtgcccctcgtgggtctcgtggtggaggggggccctaacgtggtgtccatcgtcttggaatacctgcaagaagagcctcccatccctgtggtgatttgtgatggcagcggacgtgcctcggacatcctgtcctttgcgcacaagtactgtgaagaaggcggaataataaatgagtccctcagggagcagcttctagttaccattcagaaaacatttaattataataaggcacaatcacatcagctgtttgcaattataatggagtgcatgaagaagaaagaactcgtcactgtgttcagaatgggttctgagggccagcaggacatcgagatggcaattttaactgccctgctgaaaggaacaaacgtatctgctccagatcagctgagcttggcactggcttggaaccgcgtggacatagcacgaagccagatctttgtctttgggccccactggccgcccctgggaagcctggcacccccgacggacagcaaagccacggagaaggagaagaagccacccatggccaccaccaagggaggaagaggaaaagggaaaggcaagaagaaagggaaagtgaaagaggaagtggaggaagaaactgacccccggaagatagagctgctgaactgggtgaatgctttggagcaagcgatgctagatgctttagtcttagatcgtgtcgactttgtgaagctcctgattgaaaacggagtgaacatgcaacactttctgaccattccgaggctggaggagctttataacacaagactgggtccaccaaacacacttcatctgctggtgagggatgtgaaaaagagcaaccttccgcctgattaccacatcagcctcatagacatcgggctcgtgctggagtacctcatgggaggagcctaccgctgcaactacactcggaaaaactttcggaccctttacaacaacttgtttggaccaaagaggcctaaagctcttaaacttctgggaatggaagatgatgagcctccagctaaagggaagaaaaagaaaaagaagaaaaaggaggaagagatcgacattgatgtggacgaccctgccgtgagtcggttccagtatcccttccacgagctgatggtgtgggcagtgctgatgaaacgccagaaaatggcagtgttcctctggcagcgaggggaagagagcatggccaaggccctggtggcctgcaagctctacaaggccatggcccacgagtcctccgagagtgatctggtggatgacatctcccaggacttggataacaattccaaagacttcggccagcttgctttggagttattagaccagtcctataagcatgacgagcagatcgctatgaaactcctgacctacgagctgaaaaactggagcaactcgacctgcctcaaactggccgtggcagccaaacaccgggacttcattgctcacacctgcagccagatgctgctgaccgatatgtggatgggaagactgcggatgcggaagaaccccggcctgaaggttatcatggggattcttctaccccccaccatcttgtttttggaatttcgcacatatgatgatttctcgtatcaaacatccaaggaaaatgaggatggcaaagaaaaagaagaggaaaatacggatgcaaatgcagatgctggctcaagaaagggggatgaggagaacgagcacaaaaaacagagaagtattcccatcggaacaaagatctgtgaattctataacgcgcccattgtcaagttctggttttacacaatatcatacttgggctacctgctgctgtttaactacgtcatcctggtgcggatggatggctggccgtccctccaggagtggatcgtcatctcctacatcgtgagcctggcgttagagaagatacgagagatcctcatgtcagaaccaggcaaactcagccagaaaatcaaagtttggcttcaggagtactggaacatcacagatctcgtggccatttccacattcatgattggagcaattcttcgcctacagaaccagccctacatgggctatggccgggtgatctactgtgtggatatcatcttctggtacatccgtgtcctggacatctttggtgtcaacaagtatctggggccatacgtgatgatgattggaaagatgatgatcgacatgctgtactttgtggtcatcatgctggtcgtgctcatgagtttcggagtagcccgtcaagccattctgcatccagaggagaagccctcttggaaactggcccgaaacatcttctacatgccctactggatgatctatggagaggtgtttgcagaccagatagacctctacgccatggaaattaatcctccttgtggtgagaacctatatgatgaggagggcaagcggcttcctccctgtatccccggcgcctggctcactccagcactcatggcgtgctatctactggtcgccaacatcctgctggtgaacctgctgattgctgtgttcaacaataccttctttgaagtaaaatcaatatccaaccaggtgtggaagttccagcgatatcagctgattatgacatttcatgacaggccagtcctgcccccaccgatgatcattttaagccacatctacatcatcattatgcgtctcagcggccgctgcaggaaaaagagagaaggggaccaagaggaacgggatcgtggattgaagctcttccttagcgacgaggagctaaagaggctgcatgagttcgaggagcagtgcgtgcaggagcacttccgggagaaggaggatgagcagcagtcgtccagcgacgagcgcatccgggtcacttctgaaagagttgaaaatatgtcaatgaggttggaagaaatcaatgaaagagaaacttttatgaaaacttccctgcagactgttgaccttcgacttgctcagctagaagaattatctaacagaatggtgaatgctcttgaaaatcttgcgggaatcgacaggtctgacctgatccaggcacggtcccgggcttcttctgaatgtgaggcaacgtatcttctccggcaaagcagcatcaatagcgctgatggctacagcttgtatcgatatcattttaacggagaagagttattatttgaggatacatctctctccacgtcaccagggacaggagtcaggaaaaaaacctgttccttccgtataaaggaagagaaggacgtgaaaacgcacctagtcccagaatgtcagaacagtcttcacctttcactgggcacaagcacatcagcaaccccagatggcagtcaccttgcagtagatgacttaaagaacgctgaagagtcaaaattaggtccagatattgggatttcaaaggaagatgatgaaagacagacagactctaaaaaagaagaaactatttccccaagtttaaataaaacagatgtgatacatggacaggacaaatcagatgttcaaaacactcagctaacagtggaaacgacaaatatagaaggcactatttcctatcccctggaagaaaccaaaattacacgctatttccccgatgaaacgatcaatgcttgtaaaacaatgaagtccagaagcttcgtctattcccggggaagaaagctggtcggtggggttaaccaggatgtagagtacagttcaatcacggaccagcaattgacgacggaatggcaatgccaagttcaaaagatcacgcgctctcatagcacagatattccttacattgtgtcggaagctgcagtgcaagctgagcataaagagcagtttgcagatatgcaagatgaacaccatgtcgctgaagcaattcctcgaatccctcgcttgtccctaaccattactgacagaaatgggatggaaaacttactgtctgtgaagccagatcaaactttgggattcccatctctcaggtcaaaaagtttacatggacatcctaggaatgtgaaatccattcagggaaagttagacagatctggacatgccagtagtgtaagcagcttagtaattgtgtctggaatgacagcagaagaaaaaaaggttaagaaagagaaagcttccacagaaactgaatgctagtctgttttgtttctttaatttttttttttaacagtcagaaccactaatgggtgtcatcttggccatctaaacatcatcaatttctaaaaacattttccttaaaaaattttggaaattcagacttgatttacaatttaatgcactaaaagtagtattttgttagcatatgttagtaggcttagttttttcagttgcagtagtatcaaatgaaagtgatgatactgtaacgaagataaattggctaatcagtatacaagattatacaatctctttattactgagggccaccaaatagcctaggaagtgccctcgagcactgaagtcaccattaggtcacttaagaagtaagcaactagctgggcacagtggctcatgcctgtaatcctagcactttgggaggccaaggcagaaagatagcttgagtccaggagtttgagaccagcctgggcaacatagtgataccccatctcttaaaaaaaaaaaaaaaa

SEQ ID NO: 5 - DNA, 4969 bp, Human
  MTMR10
agaaagggcctgggcggccgcactgagagctttacgcccggaggcgtcggcgctgccactggcccgcgacgggaacggggcgaaaaggcggcggcaccatgttctccctcaagccgcccaaacccaccttcaggtcctacctcctgccaccgccccagactgacgataagatcaattcggaaccgaagattaaaaaactggagccagtccttttgccaggagaaattgtcgtaaatgaagtcaattttgtgagaaaatgcattgcaacagacacaagccagtacgatttgtggggaaagctgatatgcagtaacttcaaaatctcctttattacagatgacccaatgccattacagaaattccattacagaaaccttcttcttggtgaacacgatgtccctttaacatgtattgagcaaattgtcacagtaaacgaccacaagaggaagcagaaagtcctaggccccaaccagaaactgaaatttaatccaacagagttaattatttattgtaaagatttcagaattgtcagatttcgctttgatgaatcaggtcccgaaagtgctaaaaaggtatgccttgcaatagctcattattcccagccaacagacctccagctactctttgcatttgaatatgttgggaaaaaataccacaattcagcaaacaaaattaatggaattccctcaggagatggaggaggaggaggaggaggaggtaatggagctggtggtggcagcagccagaaaactccactctttgaaacttactcggattgggacagagaaatcaagaggacaggtgcttccgggtggagagtttgttctattaacgagggttacatgatatccacttgccttccagaatacattgtagtgccaagttctttagcagaccaagatctaaagatcttttcccattcttttgttgggagaaggatgccactctggtgctggagccactctaacggcagtgctcttgtgcgaatggccctcatcaaagacgtgctgcagcagaggaagattgaccagaggatttgtaatgcaataactaaaagtcacccacagagaagtgatgtttacaaatcagatttggataagaccttgcctaatattcaagaagtacaggcagcatttgtaaaactgaagcagctatgcgttaatgagccttttgaagaaactgaagagaaatggttatcttcactggaaaatactcgatggttagaatatgtaagggcattccttaagcattcagcagaacttgtatacatgctagaaagcaaacatctctctgtagtcctacaagaggaggaaggaagagacttgagctgttgtgtagcttctcttgttcaagtgatgctggatccctattttaggacaattactggatttcagagtctgatacagaaggagtgggtcatggcaggatatcagtttctagacagatgcaaccatctaaagagatcagagaaagagtctcctttatttttgctattcttggatgccacctggcagctgttagaacaatatcctgcagcttttgagttctccgaaacctacctggcagtgttgtatgacagcacccggatctcactgtttggcaccttcctgttcaactcccctcaccagcgagtgaagcaaagcacggaatttgctataagcaaaaacatccaattgggtgatgagaagggcttaaaattcccctctgtttgggactggtctctccagtttacagcaaaggatcgcacccttttccataaccccttctacattggaaagagcacaccttgtatacagaatggctccgtgaagtcttttaaacggacaaagaaaagctacagctccacactaagaggaatgccgtctgccttaaagaatggaatcatcagtgaccaagaattacttccaaggagaaattcattgatattaaaaccaaagccagatccagctcagcaaaccgacagccagaacagtgatacggagcagtattttagagaatggttttccaaacccgccaacctgcacggtgttattctgccacgtgtctctggaacacacataaaactgtggaaactgtgctacttccgctgggttcccgaggcccagatcagcctgggtggctccatcacagcctttcacaagctctccctcctggctgatgaagtcgacgtactgagcaggatgctgcggcaacagcgcagtggccccctggaggcctgctatggggagctgggccagagcaggatgtacttcaacgccagcggccctcaccacaccgacacctcggggacaccggagtttctctcctcctcatttccattttctcctgtagggaatctgtgcagacgaagcattttaggaacaccattaagcaaatttttaagtggggccaaaatatggttgtctactgagacattagcaaatgaagactaaaatagggtgttttctgaacattttgagggaagctgtcaacttttttcctctgaattaacattgctaacctaggcgtttgaatctctaataactttatatgtaagaataatagttggaatttgcactaatatttaaaaacatgttgaatcatgcttctttcacacttattttaagagagatgtaaattttgttcctgtcctctttctgtcattacaggtctggctcttgtaaccgtgatcaaactgttcatgttgtctgctacatttttgtctccatccatttttcctaccacctcctgaaggctatctgatagtcagtcacattagcagccccaggcagcagacaacaggaaagttaggaaatttgtgtttcgtgtcatttttaggagcatctgataaaacctccagcaggttttaggaagtattcatgtatttttctggttactttctgtcgtctctaattgaactcacctgatgaaggttcagtgttctggggccagaatttatgattttagatcaccttctttggaaccttagatcactgtgttttgaaatcatgagtttgcttttaacttcatagggtcaactttaaaatgatatgcactgttaattttaaagcatttgctgcagataattaaacttagaagtgcctttgactttaggatacaaatattacagaagaaaatataatttcactttttaaaattggggtgggaaaatcccattgcatatttgaaataggcttttcatactaagcttcatagccaggagtccccagagtcttgttcctctgaaagccactggggagtggcctctggggtgctgattccacagaggtgtatgctgtagacaggagagtgccatctatgccaaaactcgccctcaaaaacaaacaaggcttgctgggaggcgtgctgggcttggccatcagtatttccagtgtggtaaactattgctggcacttccccctggaaataactaatgaggttacgagttgggcacctgcacagatgtccttctctcatagttcctaatgcttaggaatagaggagaaataaaaaaatggattctctcaaaacactgccatttgaatagcgacagaagtgctcccccagcccccaactttggacagcaaagttgaggagaatgagcagacacagttgtttgcttgatctgaatctctctaaagtaaagtatttccaaactgtgtgacaagagcctacctaccactgtagcggtcaaagctgaagcttcttacagcagtgaaacggggcaccacctcccccacactcctcattccccgcttaaaacatggatactttcaaatttgactgtttcttaaactgccatcctaagatatggaaaatttttatagtaaagtgtctagttagcttatttccttttctaaaacaagtgttttcaagataactgtattttacctttatatgtactgaatagctgtttctttttgaattatttgccttttaaaatttgataatgtctctggatataacaggacaggagttcttaaaaaatatcttaagaaattcactttatgggtaaacccaaggtttttgccaacttgttgcctagaaaataagggctagtttcagtttatacaaatagaattattaaacattttacagtccttgattagaaaccagacccaatctccttataacaccacagcgtatcctgccattgacagtgtaatcacaattctccctttttcatttagctgcttttttattattactaaatgttttggattgagcatttttccctctgtaattttcttccttcacgtttattttaactcttgtagtattttattgttgttaatttacaagtttaaaaatattaggtactattaataatggttaaaaatagaaaaatgcatatttttgtatgataatcaaatgtaaaatacttttatttttgctggacagttgttatatcatgattattgtgctacagtttattgtgcataatatgaaaaacaactatgacagccttcagtcgggccagggtgaagctgcttataccacctctgccgtcagagggacatgtggtgacagcagtggtgtggctgcacagggcgcactagagagagctcagcacccctgctgcccgccagcagagcccgtgctgagggaatgccgcacagatgctgatgcactgggtgaaatttctagtattgaacgtaaaggtgtacagtgtcttgctgttattttatgatggaaactgattttgaaaccaaaaatagctaactaactttatttaaggaaaggatattaatttgtactaacagagggtgaaagctgttcacatttgtcaacaaaatctgcttgctgcagtagtaacctcaagtggttaaaacttgatttcccgagaaaactaaaacctttgtgcctaaaattgatgacttgagttcaagtgggatgagcaagaagatgtgttatcttgttgttcaacagtattgaatgtgaaggaaattttgatggcttaataaaattccacagcgactgttaaaaaaaa

SEQ ID NO: 6 - DNA, 4835 bp, Human
  MTMR15
acagggcaccgagggaaggaggacgcgagggcagccaggccctagggagcagggagagtggctcgggctcagtcgcgtggccccaggaagaagaaattgtcgagacgaataacatgaggtcatatagaatcccacttttggtgatttcaagtcaagaaagtaaaagtaaaccattgctatctttcaccttaaatatcctgtgttttattgctcagaacatccagtttttctaatactcatgatgtcagaagggaaacctcctgacaaaaaaaggcctcgtagaagcttatcaatcagcaagaataagaaaaaagcatctaattctattatttcgtgttttaacaatgcaccacctgctaaacttgcctgccccgtttgcagtaaaatggtgcctagatatgacttaaaccggcaccttgatgaaatgtgtgctaacaatgacttcgttcaagtggatccagggcaggttggcttaataaattcaaatgtgtctatggtagatttaaccagtgttaccttagaagatgtaacacctaagaagtcaccaccaccaaagacaaatttaacccctggccaaagtgattcagcaaaaagggaagtaaagcagaagatcagtccctactttaaaagtaatgatgtggtgtgcaaaaatcaagatgagctgagaaatcgtagtgtgaaagtcatttgtttgggaagcctagcatctaaattgtccagaaaatacgtaaaggctaaaaaatcaatagataaggatgaagaatttgccggttctagtccacagagttccaaatccacagttgttaagagcctgattgataactcttcagaaattgaggacgaggatcaaattttggagaacagttctcaaaaagaaaacgtgtttaaatgtgattctctaaaggaagagtgcattcctgaacatatggtaagaggaagtaaaataatggaagccgaaagccaaaaggctacccgggaatgtgagaaatcagccctcacccctggattctcagataatgcgatcatgttattctcaccagatttcactcttaggaatacattaaagtctacttcagaagacagtcttgtaaagcaagagtgtatcaaagaagtggttgaaaaacgtgaggcatgtcattgtgaagaagtaaaaatgactgttgcttcagaagctaaaatacagctgtcagattcagaggcaaaatctcatagttctgcagatgatgcttctgcatggagtaacatccaagaggctcctctgcaggatgacagttgcttaaacaatgatatccctcacagcattcctttggagcaggggtcaagctgcaatggtcctggtcaaacaaccggtcatccttactaccttcggagtttccttgtggtgctgaaaaccgtacttgagaatgaagatgatatgttgctctttgatgagcaggagaagggaattgtaactaaattttatcagttatcagctactggtcagaagttatatgtaaggctctttcaacgtaaattaagctggattaagatgaccaaattagagtatgaagagattgccttagacttaacacctgtgattgaagaattgacgaatgcaggctttctacagacagaatctgagttgcaagaactctctgaagtgcttgaactcctttctgctcctgaactaaaatccctagccaagaccttccacttggcgaatcccaatggacagaaacagcagctggtggacgcctttctcaaattggccaaacagcgttcagtctgcacttggggcaagaataagcctggaattggtgcagtgattttaaaaagagccaaagccttggctggacagtcagtacgaatctgtaaaggccccagggctgtgttttcccgcatcttgctactgttttcgttgaccgactcaatggaagatgaagacgccgcttgtggaggtcagggacagctttcaacagtcctgttggtcaacctcggccgaatggagtttcctagttacaccatcaatcggaaaacccacatcttccaagacagagatgatcttatcagatatgcagcagccacgcacatgctgagtgacatttcttccgcaatggccaatgggaactgggaagaagctaaggagctcgctcagtgtgcaaaaagggattggaacagactgaaaaaccacccttctctgagatgccacgaagatttaccactcttcctgcggtgtttcactgttgggtggatttatacaaggattttgtctcggtttgtggaaatactgcagagacttcacatgtatgaggaagccgtcagagaacttgaaagccttttgtctcagagaatttattgtcctgacagcagaggccgatggtgggatcgactggcccttaatttacaccagcacttgaagcgcctggaaccgactatcaagtgcatcacagaggggctggcggatccggaagtcagaacgggacaccgcctttcactgtatcagcgagccgtgcgcctgcgagagtctccgagctgtaaaaagttcaagcacctcttccagcagctcccagaaatggctgtgcaagatgtgaaacacgtgaccatcacaggcaggctgtgcccacagcgtgggatgtgcaagtctgtgtttgtgatggaggccggggaggccgctgaccccaccacggtcctgtgctctgtggaggagctggcactggcccattacagacgcagcggttttgaccaggggattcatggcgaagggtccaccttcagcaccctgtatggcctcctcctgtgggacatcatcttcatggatgggattccggatgtcttcagaaacgcctgtcaggcattccccctggacttgtgcacagacagcttcttcacaagcagacgcccagcccttgaggccaggctgcagctgattcatgatgcccccgaggagagcctgcgggcctgggtggcagccacgtggcatgagcaggaaggcagagtggcttcccttgtcagctgggatcgcttcacgtctcttcagcaagctcaggatcttgtctcctgcctggggggccctgtgctcagtggtgtgtgcaggcacctggctgctgactttcgacactgtcgagggggcctccccgacctggtggtgtggaactcccagagccgtcactttaagctggtggaagttaaaggccccaatgatcgtctttcacataagcagatgatctggctggctgaactgcagaagctgggggctgaagtagaagtctgccatgtggttgcagttggagctaagagccaaagccttagctaaaagattccctacaggagaaaatggaaatgaggaggagagaaactccggtgtccccgaggtgtcggtgtggtgagggccgctggcgttgaagtacatcctgctctggcccagctccccatagcaggcctccagggggccactgcgctgttgccgcagcatcctgctcagtacgtcgacttcatcagccaggagggagagcttgtgaaaggctgtgatggagccacccaggctgatctgggcctcgggaacccagcggaagtagcacagtttccacagttttatgtgtgttccagagacacgtggcagaataacaccgtgcaggttggcgggtttggaaaaccattctctaaaatactgctccgtatcactgttctggctgtcggtttgctgagctggatctggctttggttttaatatcaatgaatttctccttggaagtaattcttggtcactgatgattccattctttaaggcagacggcattcctcttagtgtggagctgtagcttttctatacagaagagattttattatgttccggggattccctttttagaaagattgaaggatgcaatggcaaatataaactcaatactatgaaaaattaatggaatttcagcctcaaagaacattttcctcccttcctttgtgtccttattctaatcctcctcccctggaattacacttttttatgtgttgactctacctaggctgttactatcagcctgaatgggggcgggatgagagtacctcctatccactaatttgcttaaggataagttctaagacgggctagaaaaaacactagacctggtcgattctatcaagaacaatggcaaactgaacagaggcagtcaggaggccaaatgtctgattctttgttctgtacctttcagtagtctgcaaattttctaccaaaaaaaatcccaagaatttatttgggaattattaaaaaggcaaacaatgaatgttattaggacaagaatatagcagtcaggaggccatgactacatcacagccaggcggcattccctgccacagtggcggcttgaatcatcaagaaatggataaatggggctttagtaaatcaggcttgcaggctcaaagctgcaatctgcccactctcaggtactgagactttgtgggcctcagacaccaggaagaaagttgggatacagtcatttgagttaaaaagggaatgacccctcagaaacccacattagcagtgttactcttggaactgcctttacttttaacgctctctgttctgaaaaagaggtgtttggttacgtgtgagccaacatcacgttttgttagctgtgatttacctttgtccgtttaaaagacttcacggagccattctgtatacaaggtgtgctctttccaatgtagaaggggttatggaaaagggtgcgatcctttgctgtaaactggagagaccagtcccaaacagaggggaattttaagcccttctcatcacccaattggatgtttttgcttatagcaaattcctgcaaaataaataaataaatatttgcaaaact

SEQ ID NO: 7 - DNA, 2858 bp, Human
  CHRFAM7A
agccctttcccaggcggtagcgggggcagtggtgctgttgcccttttaaactgcggcttgacgggagccgcgcctcctgtcggtggagtcggttataaagggagcagccccgcaggccgccacatagctcccgccaagtcctcggtgccccttgccattttccagccgcgctcccacgagggtcacggcggcggggagaggtggagccgcgagagctcggccgggggccccgcctggtggtcgcggccatgacagcggctcgggacaggctccttttccgcgcccctcccgccggaggtgaggggaagatgtccatgtccgggttcaaggccaaaccgaagttactggcctctatcttccaggagaaccaggagccacagccgcggctcacgccccaccgcaacattaagattacaagtggacacctgagtcagcaggacctggaatcccagatgagagagcttatctacacgactcagatcttgttgtcacccccattattgacaatccaaaggtgcagaaagcactctgacaaataatgaaacaaccaccatcggttaaatttgatgcaaaaatattgcatctaccagcattttcagttccaattgctaatccagcatttgtggatagctgcaaactgcgatattgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccctgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagtgccgtggcgccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctttggcttggcgagatttgggggtgctaatccaggacagcattacacgccacaactccagtgttcccttctggctgtcagtcgtgttgcttacggtttctttgttactttaggtagtagaatctcagcactttgtttcatattctcagatgggctgatagatatccttggcacatccgtaccatcggtcagcagggccactgagtagtcattttgcccattagcccactgcctggaaagcccttcggagagctccccatggctcctcaccaccgagacagttggttttgcatgtctgcatgaaggtctacctgaaaattcaacatttgctttttgcttgtgtacaaacccagattgaagctaaaataaaccagactcactaaatcctttccaataattgactggtggaaggaaaacaaaaaacaaaaactaaaaacctcttagcttttctgcaattcaactttttatttttatttttatttctatcaaagacggtagagagaaacagcttgatgctgtttctacatcaaaaaaaaaaaaaaaaaagacagactgttggtcttactaaggatgtttttaccagcctgcctgacttctgcaaacctaccctgtcaaggagatcaaagggacgcaggtttctgtttattctgaacaagggccaggccccgcggagtgtctttggtggatcccagataactcctaggtgctgctctcagacactgaggagttgagcaaatctgttctattctgcagaacccataggacaaataagagttctactagaattaacagcccaaaagaatagctacagctaagtgaagccacttacgtgggctttaaaaaaataatgtgttagctgattcacatgcactggagttaattagtcttagaaatgtgtgcatccatacaaatgcacaacataaagtgaacatattcctaggccctttctgcctgtgtcagggccaggaagtagaggctgggaactcttctggtccccagtatggcag

SEQ ID NO: 8 - DNA, 2794 bp, Human
  FAM7A(2)
agccctttcccaggcggtagcgggggcagtggtgctgttgcccttttaaactgcggcttgacgggagccgcgcctcctgtcggtggagtcggttataaagggagcagccccgcaggccgccacatagctcccgccaagtcctcggtgccccttgccattttccagccgcgctcccacgagggtcacggcggcggggagaggtggagccgcgagagctcggccgggggccccgcctggtggtcgcggccatgacagcggctcgggacaggctccttttccgcgcccctcccgccggaggtgaggggaagatgtccatgtccgggttcaaggccaaaccgaagttactggcctctatcttccaggagaaccaggagccacagccgcggctcacgccccaccgcaacattaagattacaagtggacacctgagtcagcaggacctggaatcccagatgagagagcttatctacacgactcagatcttgttgtcacccccattattgacaatccaaaggtgcagaaagcactctgacaattccaattgctaatccagcatttgtggatagctgcaaactgcgatattgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccctgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagtgccgtggcgccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctttggcttggcgagatttgggggtgctaatccaggacagcattacacgccacaactccagtgttcccttctggctgtcagtcgtgttgcttacggtttctttgttactttaggtagtagaatctcagcactttgtttcatattctcagatgggctgatagatatccttggcacatccgtaccatcggtcagcagggccactgagtagtcattttgcccattagcccactgcctggaaagcccttcggagagctccccatggctcctcaccaccgagacagttggttttgcatgtctgcatgaaggtctacctgaaaattcaacatttgctttttgcttgtgtacaaacccagattgaagctaaaataaaccagactcactaaatcctttccaataattgactggtggaaggaaaacaaaaaacaaaaactaaaaacctcttagcttttctgcaattcaactttttatttttatttttatttctatcaaagacggtagagagaaacagcttgatgctgtttctacatcaaaaaaaaaaaaaaaaaagacagactgttggtcttactaaggatgtttttaccagcctgcctgacttctgcaaacctaccctgtcaaggagatcaaagggacgcaggtttctgtttattctgaacaagggccaggccccgcggagtgtctttggtggatcccagataactcctaggtgctgctctcagacactgaggagttgagcaaatctgttctattctgcagaacccataggacaaataagagttctactagaattaacagcccaaaagaatagctacagctaagtgaagccacttacgtgggctttaaaaaaataatgtgttagctgattcacatgcactggagttaattagtcttagaaatgtgtgcatccatacaaatgcacaacataaagtgaacatattcctaggccctttctgcctgtgtcagggccaggaagtagaggctgggaactcttctggtccccagtatggcag